(19) United States
(12) Patent Application Publication
(10) Pub. No.: US 2026/0055453 A1
(43) Pub. Date: Feb. 26, 2026

(54) METHOD FOR PARALLEL REAL-TIME SEQUENCE ANALYSIS

(71) Applicant: SEQSTANT GMBH, Teltow (DE)

(72) Inventors: Tobias LOKA, Berlin (DE); Bernhard RENARD, Berlin (DE); Henri KNOBLOCH, Berlin (DE)

(73) Assignee: SEQSTANT GMBH, Teltow (DE)

(21) Appl. No.: 18/561,502

(22) PCT Filed: May 13, 2022
(86) PCT No.: PCT/EP2022/063044

§ 371 Date: Nov. 16, 2023

(30) Foreign Application Priority Data
May 19, 2021 (EP) 21174771.2
Aug. 12, 2021 (EP) 21190984.1

(51) Int. Cl. C12Q 1/6874; C12Q 1/6876

(52) U.S. Cl. CPC C12Q 1/6874; C12Q 1/6876

(57) ABSTRACT

A method for real-time sequence analysis of DNA fragments includes providing a DNA fragments sample and connecting first and second adapter oligonucleotides to 5′ and 3′ ends of a DNA strand of the DNA fragments, respectively. A first adapter oligonucleotide includes from 5′ to 3′ a) a first flow cell binding sequence, b) a read 1 sequencing primer site, c) optionally a random sequence, and d) a sample-specific barcoding sequence. A second adapter oligonucleotide includes from 5′ to 3′ d) a sequence complementary to the sample-specific barcoding sequence, c) optionally a sequence complementary to the random sequence, b) a read 2 sequencing primer site that might be complementary to the read 1 sequencing primer site, and a) a second flow cell binding sequence. First and second adapter oligonucleotides of one kind have complementary barcoding sequences. A sequencing by synthesis process is used for sequencing the connected adapter oligonucleotides.

The invention relates to a method for real-time sequence analysis of DNA fragments, comprising i) providing at least one sample of DNA fragments for sequence analysis, ii) connecting one kind of first and second adapter oligonucleotides to the 5′ and 3′ ends of a DNA strand of the DNA fragments of the sample, respectively, wherein a first adapter oligonucleotide comprises from 5′ to 3′ a) a first flow cell binding sequence, b) a read 1 sequencing primer site, c) optionally a random sequence, and d) a sample-specific barcoding sequence, and a second adapter oligonucleotide comprises from 5′ to 3′ d) a sequence complementary to the sample-specific barcoding sequence of the first adapter oligonucleotide, c) optionally a sequence complementary to the random sequence, b) a read 2 sequencing primer site that might be (partially) complementary to the read 1 sequencing primer site, and a) a second flow cell binding sequence, wherein first and second adapter oligonucleotides of one kind have complementary barcoding sequences, and sequencing of the DNA fragments comprising the connected adapter oligonucleotides in a sequencing by synthesis process.

Preferably, the method of the invention is for parallel real-time analysis of DNA fragments from at least two sample using different kind of adapter oligonucleotides with different barcoding sequences for each sample.

Furthermore, the invention relates to the first and second adapter oligonucleotides used in the method of the invention, which can be provided in a kit and can form a (partially) double-stranded adapter through hybridization.

### BACKGROUND OF THE INVENTION

Illumina sequencing is the current state-of-the-art next-generation sequencing (NGS) technology. It can be used to investigate the genomic information contained in any type of samples, including but not limited to tissue, blood, respiratory or environmental samples. Illumina sequencing can be applied to various types of nucleic acids, including genomic DNA, cell-free DNA (cfDNA), messenger RNA (mRNA), ribosomal RNA (16S rRNA) and many others. For all RNA analyses, the RNA is usually converted into DNA before sequencing, for example by using a reverse transcriptase enzyme. The extracted DNA is fragmented into small stretches, usually of 300-800 base pairs (bp) length. As a part of the Illumina sequencing process, a specific sequencing adapter is bound to each of these DNA fragments. Afterwards, this adapter is used to bind the fragments to the Illumina flow cell and allows for attaching the sequencing primers for the sequencing by synthesis process (SBS), which is the actual Illumina sequencing approach. Fluorescent molecules linked to the nucleotides allow the identification of the DNA sequence for each of the analyzed stretches of DNA. The output of Illumina sequencing consists of data files that contain the DNA sequences of each of the sequenced fragments. The total turnaround time from sample taking to interpretable analysis results is usually at least 24-48 hours, which is a key obstacle for using NGS as a tool for time-critical clinical applications.

While different approaches have been developed to accelerate the total turnaround time of Illumina sequencing, the sequential order of sequencing and data analysis could not be overcome in a highly scalable way.

Quick et al. (2015) developed an accelerated sequencing protocol for the Illumina MiSeq that uses short reads and reduces sequencing time by shorter cycle times and a reduced number of analyzed tiles, leading to a lower number of reads and lower average sequencing quality [1]. Therefore, this approach is not scalable (as it relies on downscaling) and is not appropriate for clinical application due to the lower sequencing quality.

A different approach, called Rapid Pulsed Whole Genome Sequencing (RPS), which was published by Stranneheim et al. (2014), relies on the conversion of interim sequencing data to the human readable FASTQ Illumina file format with follow-up analysis [2]. This conversion step is time consuming and required for most pipeline analyses applications. For fast results, the analysis workflow proposed by Stranneheim et al. (2014) requires a massive reduction of analyzed targets and is only applicable for a single sample per sequencing run. This limitation remains, as the approach does not include a solution for so-called multiplexed sequencing, i.e. sequencing of several samples tagged with and identified by specific barcode sequences. Therefore, the approach does not allow for parallel clinical routine usage.

Miller et al. (2015) proposed to couple their presented specialized analysis approach based on so-called field-programmable gate arrays (FPGAs; meanwhile known as Illumina DRAGEN®) with the RPS approach to allow for analyses that are more extensive [3]. Such an approach would require the use of specialized hardware that is tailored to specific applications and comes with certain algorithmic and technical limitations. Additionally, there is no publication that demonstrates this coupled approach, and it would not solve the problem that only a single sample could be loaded into the sequencing device to analyze pulses of the first read as it would not be possible to distinguish different samples on the same flow cell. The same also accounts for published analysis approaches that enable a broad span of different analyses in real-time while still not being able to be used for more than a single sample per sequencing run [4, 5, 6, 7, 8].

Concerning the general design of sequencing adapters for Illumina sequencing, several research institutions and companies have developed methods to create Illumina-compatible sequencing libraries. The different available methods focus on different aspects of the library preparation, usually aiming at cost reduction and/or quality improvement when compared to original adapter designs provided by Illumina and official suppliers. However, the general design of the final sequences usually remained similar to the design proposal of Illumina, comprising a flow cell binding site, an index primer site, an index sequence, a sequencing primer site and (optionally) a unique molecular identifier (UMI) sequence. One of the most recent significant adapter-specific optimizations focused on the improvement of multiplexing, such that huge amounts of samples can be sequenced within a single sequencing run at the same time, thereby massively reducing the overall per-sample sequencing costs. The adapter design thereby comes with a modification of the library preparation splitting the adapter into a so-called stubby adapter and an index-specific PCR primer to reduce costs and efforts for the preparation of high amounts of samples in parallel [9]. In contrast to the invention disclosed herein, these and other methods and adapter designs described in previous literature are unsuited for parallel real-time sequence analysis approaches as the barcode can only be sequenced after the first sequencing read, regardless of the described modifications or whether a UMI is included or not.

With respect to the design of specialized sequencing adapters for Illumina sequencing, there are for example approaches to modify the adapter to solve the problem of low sequence diversity in 16S rRNA sequencing applications. These adapters are designed to compensate the sequence similarity at the beginning of reads that originates from PCR amplification steps of 16S rRNA targets. For this purpose, so-called heterogeneity spacers are frequently used that append a specific sequence of different length for each sample such that the amplified primer binding site is shifted by one position for each sample. These specialized adapters may also include an inline barcode for sample identification, which is a sample-specific sequence that is integrated into the DNA fragment instead of separately sequenced parts of the adapter as in the original Illumina sequencing protocol. An example for such an approach was published by Fadrosh et al. (2014) [10].

However, and in contrast to the invention disclosed herein, these adapter designs and the method for their synthesis are meant to solve the lack of sequence diversity in targeted sequencing approaches, e.g. 16S rRNA sequencing, and are not appropriate to be used for generalized live analysis approaches. This is because the heterogeneity spacer is similar for each sequence of the same sample, which results in diversity staying low when sequencing only few samples. Therefore, clusters of the same sample being in physical proximity on the flow cell cannot be distinguished from each other by the sequencing device, which leads to a loss of the signals of both clusters. According to Illumina, this process of assignment of signals to a specific cluster takes place in the first 4-7 cycles of each sequencing run; therefore, it is crucial to have highest possible diversity for these cycles. This also limits the combination of barcode sequences to those having a high diversity within the first few base pairs.

Finally, the existing protocols rely on a PCR step to connect the adapter sequences to the target sequences. For many applications, it is crucial to use a PCR-free approach to prevent the creation of amplification errors and artifacts.

Other sequencing adapter designs have been described in patents WO 2018/053362 A1 [11], WO 2017/223366 A1 and WO 2018/094031 A1 [13]. The adapter design described in is designed for capture-based sequencing approaches. Thereby the top (amplification) strand is combined with a bottom (blocking) strand that lacks several adapter-specific elements, such as a flow cell binding site and a sequencing primer site. Thus, the double-stranded molecule consisting of top and bottom strand differs from the novel Y-shape double-stranded design for parallel real-time sequence analysis according to the present invention. Consequently, and as previously stated for other existing methods, also in this prior art method one or more PCR steps are required for the preparation of the final sequencing library. Additionally, the context of this prior art method requires an additional index sequence upstream of the sequencing primer site to allow for multiplexed sequencing, which is not required when using the novel adapter design of the present invention. The same holds true for the approach described in [13], which uses TA-ligation to bind a linker sequence, UMI sequence and anchor sequence to the target DNA fragment in a separate step before appending the remaining parts of the sequencing adapter in a consequent indexing PCR step. Accordingly, these prior art adapters ([11], [13]) would not be suitable for a parallel real-time sequence analysis. The adapter design described by Accuragen Holding Ltd. is described in the context of cell-free DNA (cfDNA) sequencing, but while the basic structural elements required for parallel real-time sequence analysis are described, neither the order of adapter sequence elements are proposed therein, nor the Y-shaped double-stranded design according to the invention, which are required in the scope of the invention disclosed herein. Two novel sequencing technologies are currently arising that allow for real-time analysis of genomic data. The Single-Molecule Real-Time (SMRT) sequencing technology of Pacific Biosciences, for example used for their Sequel 2 device, relies on the sequencing by synthesis (SBS) approach that is also used in Illumina sequencing. However, while sequencing quality became decent over the last years, the technology is still expensive and provides only low throughput compared to Illumina sequencing.

Secondly, the sequencing technology of Oxford Nanopore (ONT; e.g., used for their MinION and PromethION devices) relies on a completely different molecular approach by measuring electrical signals for determining the correct base calls. While providing long reads and—in principle—providing high throughput devices, the sequencing quality is way lower than that of Illumina and SMRT sequencing. Additionally, for both ONT and SMRT sequencing, higher amounts of input DNA are required to prepare a sequencing library, which is often problematic for clinical applications. According, the Illumina SBS remains the gold standard sequencing approach in terms of sequence data quality when using small amounts of input DNA. However, parallel real-time analysis of multiple samples in the same flow cell using the Illumina sequencing technology has to date not been achieved.

Importantly, the sequential paradigm of wet lab (i.e., sample preparation and sequencing) and a consecutive dry lab (i.e., data analysis) of Illumina short-read sequencing leads to high turnaround times from sample arrival to analysis results. Even with fully automated sample preparation and a standard read length of 150 bp results cannot be provided earlier than 24 hours after sample taking in a theoretical best-case scenario. In practice, the time to result of current Illumina sequencing applications in a clinical setup is usually at least 36 to 48 hours. If longer reads or paired-end reads are required for follow-up analyses such as assembly or variant calling, the turnaround time can further increase to more than 48 hours.

This long duration of the overall process leads to only limited applicability of Illumina sequencing in all important fields and use cases where fast results are crucial, such as the diagnosis of respiratory-/urine tract infections (caused by bacteria, fungi or viruses), bacteremia and sepsis, the determination of M. tuberculosis and other pathogens and their drug resistances, liquor/cerebrospinal fluid analyses, transplantation diagnostics, time-critical diagnostics of autoimmune diseases, the (differential) diagnosis of genetic disorders in infants, oncology, forensics, and detection of microbial contamination in batch processes, e.g., in the production of food, paints, coatings, pharmaceuticals and others. Further applications might include to trace the biological, geographical or any other origin of a sample, the detection of genetically modified organisms, the identification of plant pathogens, the general (sample-specific) quality control of a sequencing run or the identification of an optimal time point to stop a sequencing run for cost and usage optimization when all relevant information was already obtained.

As stated above, different approaches have been described to reduce the turnaround time needed for NGS-based diagnostics. However, all these methods come with the limitation of data quantity and quality as well as the applicable analysis methods [1, 2], are only applicable for very specific types of analyses as they require the use of specialized hardware [3] or enable only the live analysis of a single sample for the first read [4, 5, 6, 7, 8]. In a clinical environment and for various other applications the implementation of live sequencing approaches requires highest possible sequencing quality and quantity, as well as an assignment of analyzed sequences to different samples from the very beginning of the sequencing run to allow real-time identification of multiple samples that are analyzed in parallel. Thereby, it is crucial that the live sequencing approach is capable to be used with a flexible number of samples per run, which can be a single sample up to several thousands of samples depending on the application, the desired genome coverage and the sequencing device used.

Although several attempts have been made, a complete solution for all technical and analytical challenges arising for the live analysis of Illumina sequencing data has not yet been developed.

In conclusion, there is a need in the art for a new adapter design that enables a live parallel/multiplex sequencing approach and that preferably also solves the problem of required high sequence diversity in the initial 4-7 sequencing cycles enabling all combinations of one or more different barcoding sequences, optimally, even when only using a single barcode. Furthermore, ideally such adapters can be used with PCR-free library preparation approaches by connecting the adapter to the DNA fragments to be sequence by different techniques, such as ligation.

### SUMMARY OF THE INVENTION

In light of the prior art the technical problem underlying the present invention is to provide sequencing adapter and a sequencing method employing such adapters that enable parallel real-time analysis of DNA sequences from more than one sample.

This problem is solved by the features of the independent claims. Preferred embodiments of the present invention are provided by the dependent claims.

The invention therefore relates to a method for real-time sequence analysis of DNA fragments, comprising
providing at least one sample of DNA fragments for sequence analysis,
connecting one kind of first and second adapter oligonucleotides to the 5′ and 3′ ends of a DNA strand of the DNA fragments of the sample, respectively,
wherein a first adapter oligonucleotide comprises from 5′ to 3′
   a first flow cell binding sequence,
   a read 1 sequencing primer site,
   optionally a random sequence, and
   a sample-specific barcoding sequence,
and a second adapter oligonucleotide comprises from 5′ to 3′
   a sequence complementary to the sample-specific barcoding sequence of the first adapter oligonucleotide,
   optionally a sequence complementary to the random sequence,
   a read 2 sequencing primer site, and
   a second flow cell binding sequence,
wherein first and second adapter oligonucleotides of one kind have complementary barcoding sequences, and
sequencing of the DNA fragments comprising the connected adapter oligonucleotides in a sequencing by synthesis process.

Preferably, the method of the invention is for parallel real-time analysis of DNA fragments from at least two samples, wherein
at least two samples of DNA fragments are provided, and
for each sample a different kind of first and second adapter oligonucleotides are connected to the 5′ and 3′ ends of a DNA strand of the DNA fragments, wherein different kinds of adapter oligonucleotides have different barcoding sequences, and
wherein the DNA fragments from the at least two samples comprising the connected first and second adapter oligonucleotides are sequenced in one reaction vessel, such as a flow cell.

In a further aspect, the invention relates to a (first) adapter oligonucleotide for parallel real-time sequencing comprising from 5′ to 3′
a first flow cell binding sequence,
a read 1 sequencing primer site,
characterized in that 3′ (downstream) from the read 1 sequence primer site there is a sample-specific barcoding sequence.

The present invention is based on the entirely surprising finding that provision of sequencing results from a sequencing by synthesis process is possible already during the sequencing process (sequencing run), even for sequences form multiple samples which are analyzed in the same flow cell, when the ends of the DNA fragments of each of the samples have been connected to adapter oligonucleotides according to the present invention. In these adapter oligonucleotides, the barcoding sequence, which is specific for each sample, is provided downstream of the read 1 sequence primer site, and therefore is read in the beginning of the first sequencing read of the sequencing by synthesis (SBS) process, before the sequence of the actual DNA fragment is being sequenced. Accordingly, it is possible to assign a sequence which is detected from an individual cluster in the sequencing chamber to a specific sample based on the detected barcoding sequence, and the following bases of the sequence can be assigned to that sample almost immediately. Herein, the terms oligonucleotide and oligo are used interchangeably.

Accordingly, due to this new arrangement of sequences in the adapter oligonucleotide, which comprise a barcoding sequence downstream from the read 1 primer site, it is possible to enable sequence analysis already during the sequencing run, and not only hours or days later once the sequencing run is finished. In classical adapter used in SBS process, the barcoding sequence (sometimes also called index sequence) is located upstream of the read 1 primer site, and the barcoding sequence is only read in a subsequent second so-called barcoding read (or index read) step using a different primer for initiation of the sequencing.

Importantly, the positioning of the barcoding sequence downstream of the read 1 primer site was non-obvious, since in the usual SBS workflow, an assignment of the sequence during the sequencing run is not possible due to the analysis/detection steps that are carried out during the process, usually using a standardized software, which is unable to detect barcoding sequences within the read 1 sequencing run. However, using different detection steps and a different sequence of signal detection and assignment steps during the read 1 sequencing run, it is possible to detect and assign a barcoding sequence already during the sequencing run, making it possible to analyze a detected sequence already during the sequencing run.

The adapter oligonucleotide according to claim 1, wherein the adapter comprises 3′ (downstream) of the sequencing primer site and 5′ of the barcoding sequence a random sequence, wherein the random sequence has preferably a length of 3-10, more preferably 4-7 nucleotides. In embodiments, the random sequence can have a length of 25, 24, 23, 22, 21, 20, 19, 18, 17, 16, 15, 14, 13, 12, 11, 10, 9, 8, 7, 6, 5, 4, 3, or 2 nucleotides.

The use of a random sequence downstream of the read 1 primer binding site and upstream of the barcoding sequence ensures a high sequence diversity of neighboring clusters in the flow cell of the SBS process. This is advantageous because the risk of neighboring clusters having highly similar sequences in the beginning of the sequence read is very low due to the introduction of this differing random sequence, even for DNA fragments of the same sample. High sequence similarity directly downstream of the read 1 primer site is problematic, because neighboring clusters cannot be differentiated clearly, which would result in the loss of the sequences from such neighboring clusters. Additionally, calibration of the sequencing device might be negatively influenced by a low sequence diversity at the beginning of a read.

The risk of high sequence similarity of neighboring clusters is increased in cases where only one or two or few samples are analyzed in a flow cell using an adapter oligonucleotide of the invention, since the barcoding sequence downstream of the read 1 primer site is identical for DNA fragments from the same sample. Accordingly, in the extreme case of analyzing only one sample in a flow cell, without a random sequence analysis of the sequencing using the adapter of the invention will be difficult. However, the more different samples (comprising different barcoding sequences within the adapter oligonucleotide of the invention) are analyzed, the lower is the risk that neighboring clusters are from the same sample and have an identical or highly similar sequence directly downstream from the read 1 primer binding site. Accordingly, in such embodiments the random sequence may be dispensable, especially if the barcoding sequences of the different samples to be analyzed are designed in a way that high sequence diversity between the barcoding sequences is ensured.

As used herein, a sample of DNA fragments is understood to be a sample comprising DNA fragments, wherein preferably the DNA fragments are preprocessed to be suitable for adapter connection to subsequently serve as a sequencing library in the method of the present invention.

Assignment of a signal to a specific cluster in the flow cell usually occurs within the first 4-7 cycles of the sequencing process. Accordingly, it is important to ensure high sequence diversity between neighboring clusters within the first 4-7 cycles. Accordingly, the use of random sequences that are 4-7 nucleotides long is particularly advantageous in the context of the invention.

An additional advantage of the use of random sequences is that they can enable the identification of duplicate reads originating from a potential library amplification or target enrichment step. In this context, the random sequence could potentially function as a unique molecular identifier to distinguish whether two or more identical reads originate from the same biological DNA molecule (being copies from an amplification of target enrichment step) or from two different molecules. This distinction can improve various types of analyses such as variant calling.

Preferably, the random sequence is composed of a random order of A, T, G and C.

In embodiments, the adapter oligonucleotide of the invention comprises between the first flow cell binding sequence and the read 1 sequencing primer site an index or a spacer sequence.

In classical (standard) SBS sequencing as offered by the company Illumina, the index sequence (sample specific barcoding sequence) is located upstream (5′) of the read 1 sequencing primer site. In the traditional SBS process using such adapters, the index/barcode is read in a second sequencing read step after the read 1 is performed. To this end, the strand that is synthetized during read 1 is washed away and a different, so-called index-read primer is hybridized to the strand for reading the index/barcode located outside (meaning closer towards the end of the DNA fragment comprising the two adapters at its ends) the binding site of the read 1 and read 2 primers of the classical adapters.

In the context of the adapter oligonucleotide of the invention, an index (which is another word for barcoding sequence) is upstream of the read 1 primer site may not be required. However, in some cases such an index sequence can be comprised.

Possible applications for an additional use of such an index sequence located upstream of the read 1 primer site, which may be an “classical” Illumina barcode, include the identification of the adapter oligonucleotides in the context of mixed sequencing, i.e. when samples in the context of this invention are sequenced with other samples (following a conventional sequencing protocol) on the same flow cell. While such a mixed sequencing approach is in principle not desirable as the live sequencing results might be affected by the other reads, the original Illumina adapter could in principle be used to improve the correct assignment of reads to the different approaches at the end of sequencing. Additionally, additional index sequences (such as classical Illumina barcodes) could be used to detect so-called carry-over contaminations, though other methods might be preferable for this application.

Alternatively, instead of an index sequence, there can be a spacer sequence, which can be short, such as 1, 2 or preferably 3, or more nucleotides, that ensure a minimal distance between the flow cell binding sequence at the end of the adapter oligonucleotide and the read 1 primer binding site, which in the context of the method of the invention hybridizes to a read 1 or 2 sequencing primer.

It can be advantageous to include a spacer between the flow cell binding sequence and the read primer binding site since hybridization of the read primer may in embodiments be hindered by a directly neighboring flow cell binding site that can be hybridized to the oligonucleotide of the flow cell surface. Therefore, the insertion of a short spacer sequence, such as a three-nucleotide TCT sequence, which is classically used by Illumina in non-multiplex applications lacking an index sequence, can be advantageous in specific embodiments of the method of the invention.

Accordingly, as used herein, it is understood that a spacer sequence can be 1, 2, 3, 4, 5, 6, 7, 8, 9, 10 or more nucleotides which are present in the adapter oligonucleotide of the invention between the flow cell binding sequence and the read 1 primer site. Preferably, the spacer is three nucleotides long. In embodiments, the spacer can function as a barcoding sequence, which can be sample specific.

In preferred embodiments, the adapter oligonucleotide of the invention comprises a spacer with the sequence TCT between the flow cell binding sequence and the read primer binding site.

In embodiments, the adapter oligonucleotide of the invention does not comprise a spacer or index sequence between the first flow cell binding sequence and the read 1 sequencing primer site.

The adapter oligonucleotides of the invention comprise read 1 and read 2 sequencing primer sites, which may also be referred to as primer binding site, sequencing primer binding sites, or sequencing primer binding sequences. These sites are sequence segments of the adapter oligos that enable binding/hybridization of sequencing primers (so-called read primers) that are used as starting points of the sequencing reads (which means starting points for the synthesis of the complementary strand) in the SBS process of the invention.

The skilled person is able to determine optimal length of read 1 and read 2 sequencing primer sites based on the published and established protocols and data. In embodiments, the primer sites are about 15-50 nucleotides long, such as 15, 16, 17, 18, 19, 20, 21, 22, 23, 24, 25, 26, 27, 28, 29, 30, 31, 32, 34, 35, 36, 37, 38, 39, 40, 41, 42, 43, 44, 45, 46, 47, 48, 49 or 50 nucleotides. Preferably, the primer sites are about 20-45 nucleotides long, more preferably about 30-40 nucleotides long, such as 34 or 40 nucleotides, as shown in the example below.

In embodiments, the sample-specific barcoding sequence has a length of at least 4 nucleotides, preferably 4-16, more preferably 8-12 nucleotides.

As used herein, a sample-specific barcoding sequence is a sequence that is unique to the adapter oligonucleotides connected to the ends of the DNA fragment/DNA molecules provided in a specific sample. In the context of the method of the invention for parallel real-time sequencing, at least two, but preferably more different samples are analyzed in the same flow cell. The sample-specific barcoding sequence distinguishes DNA fragments of one sample from those of another sample, since it is known which barcoding sequence was used for labeling the DNA fragments of a respective sample by connecting the adapter oligos of the invention. In embodiments, the barcoding sequence used for each sample is known.

The length of the barcoding sequence can be adjusted and chosen based on the desired application of the adapter oligonucleotides. In applications comprising the sequence analysis of many different samples (such as more than 20, 30, 40, 50, 75, 96, 100, 200, 300, 400, 500, 1000 or more different samples) in parallel in the same flow cells, a longer barcoding sequence can be used to ensure that enough different barcoding sequences with a high sequence diversity are available and can be provided. However, for applications where only few, such as 1 to 10 different samples, are analyzed in parallel, shorter barcoding sequences, of for example 4 or 5 or 6 nucleotides, are sufficient for differentiating between the samples.

The use of about 8-10 nucleotides is particularly advantageous, since a high number of different barcoding sequences with high sequence variation/diversity can be provided, while the barcoding sequence in not too long and sample specific sequences are detected after fewer cycles of the sequencing process.

The longer the barcoding and optionally the random sequence are, the more sequencing cycles are required to detect sample-specific signals/sequencing data from the SBS process, i.e. the sequence of the DNA fragments of the sample without the adapter sequence. Accordingly, for the adapters of the invention, it is advantageous to use short barcoding and optionally also random sequences downstream from read primer binding site, to limit the number of sequencing cycles required for detecting the random and barcoding sequence. On the other hand, depending on the respective application of the sequencing method using the adapter oligonucleotides of the invention, the length of the barcoding and optional random sequence has to be long enough to ensure the recommended or required sequence diversity. A skilled person is aware of these advantageous and disadvantageous of different lengths of the random sequence and the barcoding sequence and can adjust these according to the respective application.

In embodiments, that first adapter oligonucleotide for parallel real-time sequencing of the invention consists of from 5′ to 3′
a first flow cell binding sequence,
optionally an index or a spacer sequence,
a read 1 sequencing primer site,
optionally a random sequence,
a sample-specific barcoding sequence,
and an optional connection site.

Preferably, the adapter oligonucleotide of the invention is hybridized to a second oligonucleotide comprising from 5′ to 3′
optionally a connection site
a sequence complementary to the sample-specific barcoding sequence,
optionally a sequence complementary to the random sequence,
a read 2 sequencing primer site, (which can be non-complementary, partially complementary or fully complementary to the read 1 sequencing primer site),
optionally an index or spacer sequence, and
a second flow cell binding sequence.

In embodiments, the second oligonucleotide comprises from 5′ to 3′
a sequence complementary to the sample-specific barcoding sequence,
a read 2 sequencing primer site, (which can be non-complementary, partially complementary or fully complementary to the read 1 sequencing primer site),
and a second flow cell binding sequence.

In embodiments, that second adapter oligonucleotide for parallel real-time sequencing of the invention comprises or consists of from 3′ to 5′
a second flow cell binding sequence,
optionally an index or a spacer sequence,
a read 2 sequencing primer site (which can be non-complementary, partially reverse-complementary or fully reverse-complementary to the read 1 sequencing primer site),
optionally a sequence complementary to the random sequence of the first adapter oligonucleotide,
a sequence complementary to the sample-specific barcoding sequence of the first adapter oligonucleotide (which may also be referred to as the barcoding sequence of the second oligo of the invention),
and an optional connection site.

In embodiments, the invention relates to a partially double stranded adapter comprising the first and the second adapter oligonucleotide of the invention as disclosed herein, which are partially hybridized to each other.

In embodiments of the invention, the first and the second adapter oligonucleotide comprise corresponding sequences and sequence domains. This means, for example, that when the first oligo does not comprise an optional sequence domain, such as the spacer or index sequence, the second oligo also does not comprise a spacer or index sequence.

In embodiments, the invention relates to a kit comprising the first and the second adapter oligonucleotide of the invention as disclosed herein.

As used herein, the terms “complementary” and “complementarity” are used in reference to nucleotide sequences related by the base-pairing rules. For example, the sequence 5′-AGT-3′ is complementary to the sequence 5′-ACT-3′.

Accordingly, in embodiments the adapter of the invention is composed of or provided in form of two oligonucleotides that can hybridize to each other to form a Y-shaped, partially hybridized dimer. Herein, the oligonucleotide comprising the read 1 sequencing primer site is referred to as the first oligonucleotide of the invention, and the oligonucleotide comprising the read 2 sequence primer site is referred to as the second oligonucleotide of the invention.

Preferably, in the method of the invention the 3′ end of the first adapter oligo is connected to the 5′ end of a DNA fragment to be sequenced, and the 5′ end of the second adapter oligo is connected to the 3′ end of a DNA fragment to be sequenced.

Preferably, after connecting the two adapter oligos of the invention, each DNA strand to be sequenced comprises at its ends the first and second adapter oligo.

Furthermore, in embodiments, the second oligo comprises a read 2 sequencing primer site that enables binding of a second sequencing primer during the SBS process. The second sequencing primer is preferably different form the first sequencing primer that can hybridize to the read 1 sequencing primer site of the first oligo of the invention. In embodiments, the two sequencing primers are used sequentially during the SBS process to sequence a respective DNA fragment from both ends. In embodiments, the read 1 and read 2 sequencing primer sites should be sufficiently different from each other to ensure differential binding of the two different sequencing primers. In embodiments the read 2 sequencing primer site can be non-complementary or partially complementary to the read 1 sequencing primer site.

In embodiments, the read 1 and read 2 sequencing primer sites are fully complementary or sufficiently complementary to enable binding of the same sequencing primer.

Preferably, the second adapter oligonucleotide of the invention comprises a (random) sequence complementary to the random sequence of the first adapter oligonucleotide, or a random sequence that is similar enough to the random sequence of the first adapter oligonucleotide to enable hybridization of both adapter oligonucleotides. Accordingly, in such embodiments the first and second oligo can hybridize to each other via the barcoding sequence and the random sequence. In embodiments, the adapter oligonucleotides are hybridized to each other to form a Y-shaped structure, wherein the two oligos are bound to each other through hybridization of the complementary barcoding sequences and, if applicable, the random sequences.

In embodiments of the invention reciting complementary sequences, complementarity can be understood as being sufficiently complementary to enable hybridization. The skilled person is aware and understands the meaning of the word complementary in the context of the use of the word. In embodiments, it is preferable that the barcoding sequence of a first adapter oligo of the invention the sequence is 100% complementary to the sample-specific barcoding sequence of the corresponding second adapter oligonucleotide, not only to enable hybridization, but also to ensure that the barcodes are identical.

Also, in embodiments, the read 1 and read 2 primer binding sites of the two oligos may be partially or fully complementary and can therefore be at least partially included in the double stranded part of the hybridized adapter oligos.

However, in embodiments the sequencing primer sites are not complementary or cannot hybridize with each other, or at least parts of the two sequencing primer sites cannot hybridize with each other.

In embodiments, the non-hybridizing parts of the two adapter oligonucleotides of the invention comprise the first and second flow cell binding sequences of the first and second oligonucleotide. Furthermore, the optional index or spacer sequences of the two oligo are non-complementary and/or do not hybridize to each other. Furthermore, in embodiments the non-hybridizing parts can also comprise the respective sequencing primer binding sites (either fully or partially).

Preferably, the first and second flow cell binding sequence of the first and second oligonucleotide are different, so that they allow differential binding/hybridization to two different oligonucleotides that are fixed on the surface of a flow cell (flow cell oligonucleotide). Preferably, the first flow cell binding sequence is suitable for hybridization to a first flow cell oligonucleotide, and the second flow cell binding sequence is suitable for hybridization to a second flow cell oligonucleotide. As used herein, “suitable for hybridization to a flow cell oligo” comprises both sequences that are (sufficiently) complementary to a sequence of a flow cell oligo to enable hybridization to the flow cell oligo (in other words, sufficiently similar to the complementary sequence of a sequence of a flow cell oligo to enable hybridization to the flow cell oligo), and sequences that are (sufficiently) identical to a sequence of a flow cell oligo so that hybridization of the complementary sequence of the flow cell binding sequence can hybridize to the corresponding flow cell oligo.

In embodiments, the first and second oligonucleotide can comprise a connection site at the 3′ and 5′ end, respectively.

In embodiments of the invention, the first adapter oligonucleotide comprises at its 3′ end a connection site.

In the context of the present invention, it is understood that a connection site is the chemical entity of an adapter oligonucleotide that is connected to a DNA fragment of a sample in the context of the method of the invention. As disclosed herein, the adapter oligonucleotides of the invention are connected to DNA fragments comprised in a sample. Connection of the oligonucleotide can occur by various techniques known to the person skilled in the art that are commonly used to connect or introduce adapter sequences or end sequences to the ends of DNA fragments. For example, ligation of the oligonucleotide can be performed using (partially) double stranded oligonucleotide adapters and double stranded DNA fragments of the sample, for example by TA ligation.

In embodiments, the first adapter oligonucleotide comprises a T nucleotide at its 3′ end. In embodiments, the second oligonucleotide is phosphorylated at the 5′ end. In embodiments, the first adapter oligonucleotide comprises a T nucleotide at its 3′ end and the second oligonucleotide is phosphorylated at the 5′ end, wherein in embodiments where the first and second oligo are hybridized to each other to form a Y-shaped, partially double stranded molecule, the T at the 3′ end of the first oligo forms a one-nucleotide overhang, and the nucleotide at the 5′ end of the second oligonucleotide is phosphorylated. Such embodiments are particularly suited for TA ligation as a method for connecting the adapter of the present invention to the end of a DNA fragment comprised in a sample.

For example, in case of TA ligation, there should be a one-nucleotide T-overhang on the 3′-end of the double stranded end of the adapter to be connected to the end of a double-stranded DNA fragment. The 5′-end of the opposite strand of the double-stranded adapter should be phosphorylated. Accordingly, the end of the double-stranded adapter to be ligated comprises on the 3′-end a T-overhang, while the opposing 5′-end is phosphorylated, and the T-overhang together with the 5′-phosphorylation represent the connection site of the adapter suitable for TA ligation.

In other embodiments, the adapter oligonucleotides of the invention may be designed in a way that at the end to be connected to the DNA fragments (herein also called “connecting end” of the adapter, corresponding to the 3′ end of the first oligo and the 5′ end of the second oligo) there is a restriction enzyme recognition site that can be cleaved by the respective restriction enzyme when the two oligos are hybridized to each other, resulting in a characteristic sticky end at the connecting end of the adapter, which can be useful for connecting the adapter to the DNA fragments of the sample. Accordingly, the restriction enzyme recognition site at the ligation end, or the resulting sticky end after restriction, can be referred to as a connection site in the sense of the present invention.

Also, it is possible to synthetize the first and second adapter oligos of the invention so that at the connecting end of the dimeric adapter there is a specific overhang sequence.

In embodiments, the adapter oligonucleotides can be connected to the DNA fragments through tagmentation, which is well established process, in which double-stranded DNA is cleaved and tagged with adapters.

Further kinds of connections sites can be envisioned by the skilled person, depending on the technique used for connecting the adapter to the DNA fragment.

In embodiments, amplification based connection can be performed, wherein the first and/or second adapter oligonucleotides (or oligos that are complementary to the first and/or second adapter oligo) are used as amplification starting points/primers that amplify the DNA fragments of the sample and thereby incorporate sequences at that (5′-)end of the resulting amplified DNA strand. For example, an adapter oligonucleotide of the invention may comprise at its 3′-end a connection site that is a sequence that is sufficiently complementary to a sequence (preferably a sequence at the 3′-end) of one or more or all DNA fragments of the sample, and the adapter oligonucleotide hybridizes to a DNA fragment (or to the corresponding strand of a double-stranded DNA fragment) and is subsequently elongated, so that a DNA strand is synthetized that is complementary to the DNA fragment (or to the corresponding strand of a double-stranded DNA fragment) and comprises at its 5′ end the sequences of the adapter oligonucleotide of the invention. The skilled person is aware of such amplification-based techniques used for introducing adapter sequences into DNA fragments of a sample and can apply these techniques in the context of the present invention. Accordingly, suitable connections sites can be included in the oligonucleotide adapters of the invention.

Furthermore, the connection sites may comprise or be composed of complementary sequence stretches at the 3′ and 5′ ends of the respective first and second oligos of the invention. In embodiments the first and second oligonucleotide can comprise connection sites that are complementary or partially complementary to each other.

In one aspect, the present invention relates to a method for real-time sequence analysis of DNA fragments, comprising
providing at least one sample of DNA fragments for sequence analysis,
connecting one kind of first and second adapter oligonucleotides of the invention to both ends of the DNA fragments of the sample, wherein the adapter oligonucleotides of one kind differ only with respect to the optional random sequence, and
sequencing of the DNA fragments comprising the connected adapter oligonucleotides in a sequencing by synthesis (SBS) process.

In SBS sequencing approaches such as Illumina sequencing, a sample is prepared for the SBS process by isolation of DNA (or any other appropriate nucleic acid) and a library preparation protocol designed for the given type of nucleic acid and application. The library preparation usually includes the fragmentation of DNA and the binding of sequencing adapters to the resulting fragments. Once the sequencing library is prepared, it is loaded to the sequencing device.

The DNA extraction and library preparation steps performed in the method of this invention are similar to the DNA extraction and library preparation of standard Illumina sequencing applications and can be performed with commercially available kits, with the only difference that the adapter oligonucleotides described in this invention are used during library preparation instead of the standard Illumina adapter oligonucleotides to allow for parallel real-time sequencing.

When loaded to the sequencing device, the single DNA molecules in the sequencing library are bound to the flow cell and amplified via a process called bridge amplification to create clusters of identical DNA molecules. This is necessary to produce fluorescent signals during SBS that are strong enough to be identified. After bridge amplification, the reverse strands are washed away and the read 1 primer is bound to start the SBS process.

The SBS process consists of a specified number of sequencing cycles. In each cycle, one single dNTP is added to the synthesized strand which is complementary to the next nucleotide of the forward strand being sequenced. The nucleotide is identified via a specific fluorescent blocking group which is removed after the signal was recorded to enable binding of the next dNTP in the following cycle. All these steps of the method of the invention are similar to the standard Illumina sequencing procedure.

However, in the standard Illumina sequencing approach, primer binding and the SBS process are repeated for the read 1, index 1, index 2 and read 2 (for paired-end sequencing). Only afterwards, demultiplexing and file conversion are executed by a program delivered by the manufacturer. The resulting files can then—after sequencing was finished—be used for data preprocessing (usually including, e.g., low quality filtering, low complexity filtering, host removal, etc.), data analysis and data postprocessing (e.g., including data integration and visualization). Only after all these steps, the results are available. Also, sample-specific quality control steps, including average base call quality, number of valid reads, average length of reads, etc. can only be performed after assigning all reads to the corresponding sample via demultiplexing.

In the context of the present invention, the term “data analysis” comprises data preprocessing, data analysis, data postprocessing, and sample-specific quality control.

In contrast, in the workflow of this invention, analysis of the data is executed in parallel to the sequencing process, i.e. while the sequencing process is ongoing/during the sequencing process. This is possible thanks to the design of the adapter oligonucleotides of this invention that are used during the library preparation step. Thereby, the random sequence is sequenced as the first part of the read 1 SBS process and is designed to enable proper cluster identification performed by the Illumina software. However, the random sequence is only a preferred feature, since in embodiments of the invention sequencing several samples in the same flow cell at the same time the barcoding sequence of the different sample may provide sufficient sequence diversity.

The sample-specific barcoding sequence is preferably sequenced as the second part of the read 1 SBS. This region is included in the first 25 base pairs which are used for calibration and quality filtering by the Illumina software and, most importantly, allows to perform demultiplexing, i.e. the assignment of reads to the corresponding samples, as the first part of the analysis which is performed in parallel to the sequencing of read 1.

The third part of the read 1 SBS is the sequence of the analysed DNA molecule. Thanks to the previously performed demultiplexing performed by the real-time analysis software, it is possible to run sample-specific analysis steps in parallel to the sequencing process (real-time analysis) which is not possible with the standard Illumina sequencing workflow. In embodiments, this real-time analysis includes all the data preprocessing, data analysis, data postprocessing and quality control steps which would be executed after demultiplexing and file conversion after the sequencing run finished in standard Illumina sequencing.

In embodiments, real-time analysis includes one or more of data preprocessing, data analysis, data postprocessing and quality control steps which would be executed after demultiplexing and file conversion after the sequencing run finished in standard Illumina sequencing.

This combination of data preprocessing, analysis, postprocessing and quality control in real-time analysis requires a very different analysis approach than with standard analysis workflows, as it is not possible to execute all analysis steps in a consecutive manner. Instead, preferably all steps are executed in parallel for all reads and extend results from previous sequencing cycles with new incoming data. Thus, the analysis performed in the context of this invention is a new conceptual approach which is complex to design and implement in an efficient way.

An additional preferred adaption in the workflow of this invention compared to standard Illumina sequencing workflows is that the separate index 1 read as well as index 1 primer provision and binding in the SBS process is no longer needed to be performed, as the barcode used for sample assignment is included in the read 1 sequence information. This adaption leads to additional time savings which is relevant in the context of real-time sequencing applications, for example in point-of-care applications.

In summary, the major adaptions in the SBS workflow compared to standard Illumina sequencing include
(1) The use of the adapter oligonucleotides of this invention during library preparation
(2) The random sequence of the adapter oligonucleotides of this invention being placed at the beginning of read 1 to be used for cluster identification by the Illumina software
(3) The sample-specific barcode sequence of the adapter oligonucleotides of this invention being sequenced after the random sequence and before the sequence of the DNA fragment to be analysed, enabling demultiplexing at the beginning of read 1
(4) Demultiplexing being executed in parallel to the sequencing of read 1, immediately after the base calls for the first cycles (usually cycles 1-25) are written by the sequencing device. The demultiplexing is integrated in the analysis method of this invention; the demultiplexing software delivered by the manufacturer (Illumina) cannot be used in this setup without major adaptions.
(5) Data preprocessing, data analysis, data postprocessing and sample-specific quality control being executed in a novel parallelized approach instead of a consecutive manner. Analysis is continuously performed in parallel to the sequencing procedure, producing results while the sequencing machine is still running.
(6) Separate sequencing of index 1 and index 2 with additional primers is not necessary, leading to additional time savings in the overall workflow.

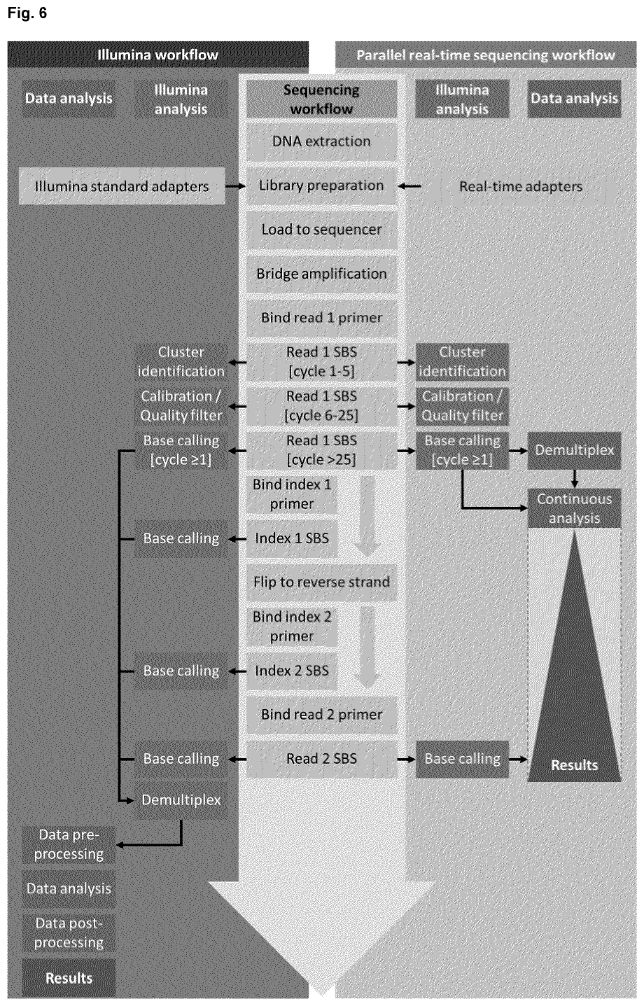

Concluding, the method of the invention includes the following workflow steps:
(1) Extraction of DNA from a sample (e.g., using commercially available kits; for example, Qiagen QIAamp DNA Microbiome Kit for isolation of bacterial microbiome DNA from mixed samples)
(2) Library preparation using the extracted DNA of one or more samples, and using the adapter oligonucleotides of the invention (e.g., using commercially available kits; for example, IDT Lotus DNA Library Prep Kit for enzymatic fragmentation and ligation-based adapter binding)
(3) Loading the sequencing library including one or more samples prepared according to steps 1 and 2 to an Illumina sequencing device, for example using an Illumina MiSeq and the Illumina MiSeq Reagent Kit v3 (600-cycle).
(4) Start the sequencing run. The sequencing device creates clusters via Bridge Amplification.
(5) After Bridge Amplification is finished, the sequencing device binds the read 1 primer to start the SBS process.
(6) The sequencing device starts the SBS process, sequencing the first few cycles needed for cluster identification (usually at most 7 cycles; cycles 1-5 in FIG. 6). The sequence information produced in these cycles preferably contains the optional random sequence of the adapter oligonucleotide of the invention.
(7) The sequencing device continues the SBS process for additional cycles needed for calibration and quality filtering (usually until cycle 25; cycles 6-25 in FIG. 6). The sequence information produced in these cycles preferably contains the sample-specific barcode of the adapter oligonucleotide of the invention.
(8) After finishing calibration and quality filtering, base calling is performed for all previous cycles and the data is written in a raw base call file format. The data analysis part of the invention runs in parallel, performs demultiplexing based on the written sequencing data and starts the continuous analysis. Thereby, all preprocessing steps, analysis steps and postprocessing steps are executed in a parallelized manner allowing for efficient extension of interim results and interaction between different analysis steps using a novel conceptual real-time analysis approach. The sequencing device continues the SBS process for the remaining cycles according to the specified read length, e.g. until cycle 301 when using Illumina MiSeq Reagent Kit v3 (600-cycle). After each cycle, new base call files are produced and analyzed with the data analysis part of the invention. Real-time results are updated continuously or in intervals. For single-end sequencing, the workflow ends with writing the base call files of the last sequencing cycle, extending analysis for the new sequence information and writing the final results. For paired-end sequencing, the workflow continues with the following steps.
(9) Sequencing of the Illumina index 1 is not required (but can optionally be included, if desired). The DNA molecules of all clusters are flipped via a single bridge synthetization step to prepare sequencing of the reverse strand.
(10) Sequencing of the Illumina index 2 is not required (but can optionally be included, if desired). The sequencing device binds the read 2 primer to start the SBS process of the reverse strand.
(11) The sequencing device performs the SBS process for read 2. As for read 1, the first written base call files include the optional random sequence and the sample-specific barcode of the oligonucleotides of the invention. This information can be ignored (because the clusters have already been assigned to the corresponding samples) or used to confirm correct sample assignment. The sequencing device continues the SBS process for the remaining cycles according to the specified read length, e.g. until cycle 301 when using Illumina MiSeq Reagent Kit v3 (600-cycle). After each cycle, new base call files are produced and analyzed with the data analysis part of the invention. Real-time results are updated continuously or in intervals. After sequencing and analysis of the last sequencing cycle, the final results are written.

The full workflow for parallel real-time sequencing is illustrated in FIG. 6, including an extensive comparison to the standard Illumina sequencing workflow.

In preferred embodiments, the at least one sample of DNA fragments for sequence analysis comprises double stranded DNA fragments. However, for connecting the adapter oligos to the DNA fragments, the DNA fragments may also be provided in single stranded form, or the dsDNA fragments are converted to single stranded fragments in the connecting process, for example by melting. The sample may be, in embodiments, fragmented genomic DNA of a subject.

However, the sample can be a sample comprising DNA that is useful for the diagnosis of a medical condition, such as an infection and related antimicrobial resistances, useful for the analysis of the microbial composition in a sample, useful for the diagnosis or prognosis of an autoimmune disease or a transplant rejection reaction or a genetic disorder or cancer. In embodiments, the method of the invention can be used for the detection of a microbial contamination of a sample, such as a food sample (or any other batch process). Also, the method of the invention can be used for a forensic or hygiene analysis of samples by analyzing the nucleic acid composition of the sample.

In the context of the invention, the connecting of the adapter oligonucleotides occurs through connection sites of the adapter oligo, which are preferably at the 3′ end of the first oligo and the 5′ end of the second oligo. In embodiments, the connecting occurs through the connecting end of a Y-shaped adapter of the invention that is composed of a first and second oligonucleotide of the invention.

In embodiments, the connecting of the adapter oligonucleotides occurs via ligation (using DNA ligases, such as preferably a T4 ligase or other known ligases commonly used in molecular biology applications), amplification, tagmentation or others or combinations thereof.

As used herein, connecting the adapter oligonucleotides to the DNA fragments of a sample may also be referred to as “labeling” of the DNA fragments of the samples, since the sample specific barcoding sequence of the sample specific adapter oligonucleotides represents a sample specific label. Accordingly, DNA fragments that have been connected to sample specific adapters may be referred to as labeled DNA fragments. Furthermore, the terms “barcoding sequence”, “index sequence”, “barcode” and “index” are used interchangeably.

In the context of the method of the invention, the DNA fragments of one sample are connected to adapter oligonucleotides that comprise the same barcoding sequence, so that all fragments of the sample comprise the same barcoding sequence after connecting of the adapter oligonucleotides.

Accordingly, in embodiments of the method of the invention DNA fragments from multiple samples could be pooled after connecting the adapter oligonucleotides with sample specific barcoding sequences to the DNA fragments, and the subsequent sequencing of the DNA fragments of multiple pooled samples can occur in the same flow cell (in the same lane of the same flow cell, meaning in the same reaction vessel).

The use of adapter oligonucleotides comprising a random sequence downstream of the sequencing primer binding sites is advantageous to ensure that there is sufficient sequence variation at the beginning of each sequencing run even if DNA fragments of only few samples or even one sample are analyzed in one lane. The random sequence, which can differ for adapter oligonucleotides that are connected to the DNA fragments of the same sample, and which is preferably the only sequence difference for the different adapter oligonucleotides used for the same sample, ensure, that during the SBS process, signals from different clusters that are located very close to each other on the surface of the reaction vessel (flow cell or lane of the flow cell) can be differentiated. In embodiments, 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, 1000, 1100 or more different first adapter oligonucleotides that differ only with respect to the random sequence are used for connecting them to DNA fragments of the same sample. In embodiments, the random sequences of one kind of adapter oligonucleotide are synthetized/generated randomly leading to a high number of different random sequences for one kind of first (and second) adapter oligonucleotide. For example, in case of a random sequence of a length of 5 nucleotides, 4\hat{ }5=1024 different random sequences and therefore 1024 different variants of the first kind of adapter oligo may be generated and used in the method of the invention.

However, if a sufficiently high number of different samples that are labeled with adapters with different barcoding sequence are analyzed in the same reaction vessel, the random sequence may be dispensable since the barcodes are different for DNA fragments from different samples, and it is unlikely that clusters of DNA fragments from the same sample are located next to each other in the reaction vessel.

Preferably, in case of parallel analysis of labeled DNA fragments from different samples is performed, the adapters used for labeling the DNA fragments from different samples differ in their barcoding sequence and optionally in the random sequence but have identical sequencing primer sites and flow cell binding sequences. This enables parallel sequencing of DNA fragments from different samples in the same reaction vessel using the same flow cell oligos and sequencing primers.

In the context of the method of the invention, after connecting the DNA fragments of one sample to oligonucleotides of the present invention, the DNA fragments are sequenced in a sequencing by synthesis (SBS) process. The SBS process has been extensively described in the art and is performed in a flow cell, which is a suitable reaction vessel of SBS. In embodiments, the flow cell can be subdivided into different lanes, so that each lane of the flow cell represents a separate reaction vessel.

SBS comprises different process steps and many different variations of this process have been described in the art and are known to the skilled person. In the following a preferred example of SBS is explained in more detail. SBS uses a DNA fragment library, wherein the DNA fragments comprise at their ends suitable adapter sequences that enable hybridization of the DNA fragments to flow cells oligos that are fixed on the surface of the flow cell. In the context of the present invention, the DNA fragment library is established by connecting the adapter oligonucleotides of the invention to the DNA fragments of a sample.

After this connecting step, the labeled DNA fragments of one or more samples are added to a reaction vessel/flow cell comprising the two different flow cell oligos that are complementary to the flow cell binding sequences of the adapter oligonucleotides of the invention and the DNA strands are bound to the flow cell surface through hybridization to the flow cell oligos. This step is the binding of the labeled DNA strands to the flow cell/reaction vessel.

Subsequently, cluster generation for the bound DNA fragments is performed via bridge amplification. Therein, the flow cell oligos are used as primers for synthetizing DNA strands that are complementary to the initially bound strand. This process is enabled by bending of the DNA strands resulting from the elongation of the flow cell oligo and hybridization of the sequence at their 3′ end comprising a flow cell binding sequence to the second kind of flow cell oligo and so on. Bridge amplification results in clonal amplification and cluster generation for each bound DNA fragment in the flow cell. Each cluster comprises copies/clones of the forward and reverse strand of a single DNA molecule of the sample which are fixed on the flow cell via the first and second flow cell oligo, respectively.

After cluster generation and clonal amplification, the reverse strands are removed from the flow cell so that only forward strands are present in each cluster. Also, the 3′ ends of the strands are blocked to prevent unwanted priming in the following sequencing process.

Subsequently, sequencing is performed by adding a first sequencing primer that binds/hybridizes to the read 1 sequencing primer site of the forward strand. Subsequently, a polymerase adds a fluorescently labeled nucleotide to the 3′-end of the read 1 sequencing primer. Only one base is able to be added per round due to the fluorophore acting as a blocking group; however, the blocking group is reversible. Using four different fluorophores with distinguishable emission (one for each of the four bases (A, T, C, G), the sequencer records which base was added for each cluster of the flow cell during each round/sequencing cycle. Alternative labeling strategies using only two or a single fluorophore have been described and can also be used in the context of the invention. Once the color is recorded the fluorophore is washed away and another dNTP is washed over the flow cell and the process is repeated.

During classical Illumina SBS process, the full sequencing process consists of two different types of reads, sequence reads containing the genomic information of the sample and index reads that are used for sample identification. In single-end sequencing, the first sequence read is followed by an index 1 sequence. The index 1 sequence can only be sequenced after finishing sequencing of the first sequence read and uses a specific index read primer. Therefore, the single-end sequencing process consists of a first sequence read and an index 1 read that can only be sequenced in this specified order and thereby deliver information for sample assignment only at the end of the sequencing process. In paired-end sequencing, the first sequence read is followed by an index 1 sequence, an optional index 2 sequence and a second sequence read. Sequencing of the first read sequence and index 1 sequence works in the same way as previously described for single-end sequencing. An additional index 2 primer can then be used to sequence a second index read (dual index). Sequencing of the index 2 sequence can be omitted (single index). As the last step, the second sequence read is sequenced using a read 2 sequencing primer on the reverse strand that is constructed in a single bridge resynthesization step. Therefore, the paired-end sequencing process consists of a first sequence read, an index 1 read, an optional index 2 read and a second sequence read in this specified order and thereby deliver information for sample assignment only after finishing sequencing of the first sequence read and one (single index) or both (dual index) index sequences.

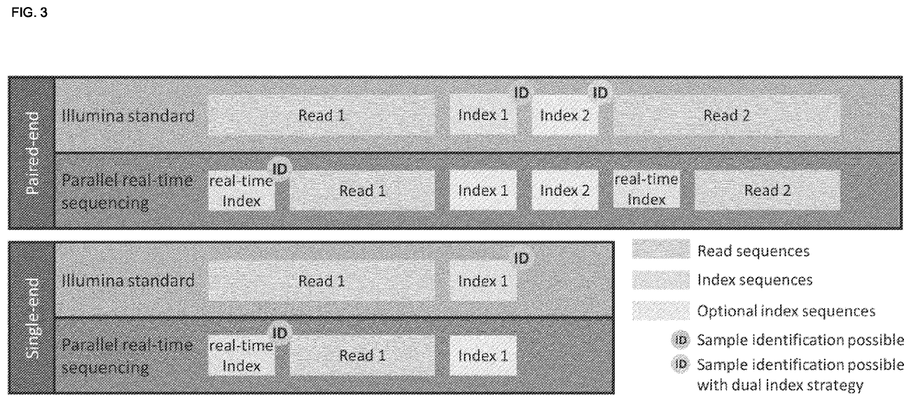

In the context of the present invention, due to the different location of the barcoding sequence downstream (3′) of the read 1 sequencing primer site of the first oligonucleotide, it is now possible to detect the barcoding sequence within the first, early cycles of the read 1 sequencing step of the SBS process as illustrated in FIG. 3. This enables assignment of signals generated by a specific cluster to a specific sample already during the sequencing process. Accordingly, it is possible to use the detected sequencing data already during the sequencing run for sequence analysis and to detect sequences of interest in the different samples that are analyzed in parallel in the same reaction chamber/flow cell.

This is a fundamental advantage in comparison to classical SBS protocols, in which the sample specific barcoding sequence (index sequence) is only detected after the first sequencing run in a separate read step. Sequence analysis that is performed already during the sequencing process is called real-time analysis, since the sequencing results are available very shortly after the actual sequencing reaction is performed and the user can get the results in “real time” while the reaction is running. In contrast, classical SBS processes as performed by standard Illumina technology can only be analyzed after the whole SBS process has been finished (single-end sequencing) or after the first sequence read and all index reads have been fully sequenced (paired-end sequencing).

In a preferred embodiment, the method for real-time sequence analysis of DNA fragments of the invention is used for parallel real-time analysis of DNA fragments from at least two samples,
wherein at least two samples of DNA fragments are provided, and
wherein for each sample a different kind of adapter oligonucleotides are connected to both ends of the DNA fragments, wherein different kinds of adapter oligonucleotides have different barcoding-sequences, and
wherein the DNA fragments from the at least two samples comprising the connected adapter oligonucleotides are sequenced in one reaction vessel, such as a flow cell.

It is a great advantage of the method of the present invention that using adapter oligonucleotides of the present invention with different barcoding sequences for each sample enables real-time analysis of the DNA sequences of the fragments from each sample during the sequencing reaction, even if the DNA fragments of the different samples are pooled and analyzed in the same flow cells. The innovative combination and assembly of sequence segments in the adapter oligonucleotides with the barcoding sequence of the first adapter oligo being located 3′ of the read 1 sequencing primer site and a corresponding arrangement in the second adapter oligonucleotide enables detection of the barcoding sequence already during the early cycles in the beginning of the first sequence read of the SBS process. Accordingly, the detected sequences can already be assigned to a specific sample during the sequencing run, enabling sample specific sequence analysis in real-time during the sequencing process. This has previously not been possible, because in known parallel sequencing reactions the barcoding sequence is only detected in a subsequent sequencing reaction (often referred to as index read) that is performed after the read 1 sequencing step.

The arrangement of the sequences is surprising, since positioning a barcoding sequence 3′ from the read 1 primer leads to a later detection of the sample specific nucleic acid sequence. Accordingly, more sequencing cycles are required to analyze the same sample specific sequence length. Furthermore, in embodiments where only one or few samples are analyzed in one flow cell, the sequence diversity at the beginning of the read 1 sequencing read would have been expected to be too low to distinguish neighboring clusters, since fragments from the same sample have identical barcodes that are read at the beginning of the run. However, in the context of the present invention, this problem can be circumvented by parallel analysis of multiple samples with different barcodes and/or by incorporating the random sequences in the adapter oligonucleotides. Accordingly, based on the present disclosure a skilled person can ensure sufficient sequence diversity of neighboring clusters at the beginning of the read 1 run although the barcoding sequence is located downstream of the read 1 primer site.

In embodiments, the method of the invention comprises real-time data analysis during the sequencing (SBS) process. In embodiments, the data analysis steps are performed by a computer program, which may be provided on a computer readable medium.

Preferably, the data analysis during the sequencing process comprises one or more of the following data analysis and/or processing steps:
the assignment of sequencing reads to cluster in the flow cell during the initial 3-10 cycles of the sequencing process, preferably based on the detected random sequence of the adapter oligonucleotide;
the assignment of preferably all sequencing reads in the flow cell to the corresponding sample of DNA fragments based on the detected sample-specific barcoding-sequence;
data preprocessing, and/or data post-processing steps, such as filtering of low quality reads, trimming of low quality ends, filtering of low complexity reads, removal of duplicates, filtering of host reads and contamination, application of Illumina filter files, evidence level calculation of results, positional peak removal, report summary, and/or calculation of quality metrics
provision of sample-specific data analysis results during the sequencing process, for example with respect to the presence of one or more specific DNA sequences in the sample;
sample-specific, optionally dynamic and/or interactive, adaption of analysis parameters to optimize computations for specific types of samples, organisms, protocols, and others;
evaluation of the reliability and completeness of real-time analysis results (i.e., results being reported before the end of the sequencing process) using algorithmic and statistical methods, learning-based approaches, artificial intelligence and/or combinations of these;
editing of the raw sequencing data including the removal or correction of sequence information in the original base call files, e.g. for correcting detected sequencing errors and/or removing human reads from the raw sequencing data, for example to comply with data protection standards;
and/or
the sample-specific visualization of analysis results during the sequencing process;
wherein preferably the data analysis is performed by a computer program.

The analysis steps listed in this embodiment are optional and an analysis in the context of the present invention can comprise one or more of these steps, which can be combined depending of the requirements of a respective analysis.

In embodiments, the data analysis during the sequencing process comprises
the assignment of (preferably all) sequencing reads in the flow cell to the corresponding sample of DNA fragments based on the detected sample-specific barcoding-sequence;
provision of sample-specific data analysis results during the sequencing process, for example with respect to the presence of one or more specific DNA sequences in the sample;
evaluation of the reliability and completeness of real-time analysis results (i.e., results being reported before the end of the sequencing process) using algorithmic and statistical methods, learning-based approaches, artificial intelligence and/or combinations of these;
editing of the raw sequencing data, e.g. correcting detected sequencing errors and/or removing human reads from the raw sequencing data, for example to comply with data protection standards;
and/or
the sample-specific visualization of analysis results during the sequencing process;
wherein preferably the data analysis is performed by a computer program.

Certain preferred embodiments of the method of the invention comprise in the data analysis the evaluation of the reliability and completeness of real-time analysis results (i.e., results being reported before the end of the sequencing process) using algorithmic and statistical methods, learning-based approaches, artificial intelligence and/or combinations of these.

This analysis step is particularly advantageous, since with known sequencing methods of the state of the art one cannot make any statement about the reliability of preliminary results and therefore a separate evaluation of correctness may be necessary. In contrast, the method of the invention enables a real-time evaluation of the reliability and/or correctness of the acquired data.

Furthermore, in such embodiments it is possible to predict the completeness of the results already during the sequencing run, meaning while the sequencer is generating data, and one could in principle abort the sequencing process, for example once sufficient data for the desired result have been acquired. This can shorten the duration of the overall workflow and would save time and resources.

In embodiments, the SBS process of the method of the invention comprises only a single sequencing read starting from the read 1 sequencing primer site (single-end sequencing). In a further embodiment, the SBS process of the method of the invention comprises only two sequencing reads starting from the read 1 sequencing primer site and the read 2 sequencing primer site (paired-end sequencing). Preferably, the method of the invention does not comprise separate index sequencing reads as required in classical SBS processes as used by Illumina.

The sequencing workflow of the invention, compared to classical SBS processes as used by Illumina, comes with several adaptions. First, in the context of data analysis, the conceptual approach of data preprocessing, data analysis and postprocessing was changed from a classical linear approach to a parallel execution of all analysis steps which is necessary in the context of the invention. In conventional Illumina sequencing data analysis, all data processing and analysis steps are for each read executed in a linear manner. For example, an analysis workflow including low complexity filtering, low quality trimming, human host removal and short read alignment steps, all these steps are applied one after each other in the specified order for a complete specific read (while, of course, parallelization is possible within a single step and/or for different reads).

In the continuous data analysis of the invention, however, all steps need to be executed in a parallelized manner to allow for efficient extension of analysis results with ongoing sequencing. This leads to non-trivial interaction between different steps of the analysis; for example, the main analysis step (i.e., short read alignment in the example given) needs to know about interim filtering and/or trimming decisions for a given sequencing cycle, and it must be considered that these interim decisions of the other modules might change for future cycles. These complex dependencies between different analysis steps are resolved by the real-time data analysis method of the invention.

Additionally, as the data analysis approach of the invention includes a demultiplexing step using the sample-specific barcodes of the adapter oligonucleotides of the invention, sequencing of the index 1 and index 2 is no longer required. Additionally, the separate demultiplexing and file conversion steps usually executed by a manufacturers software is no longer required as demultiplexing is already performed in the scope of the continuous analysis during sequencing.

Data conversion is no longer needed as the raw base call files written by the sequencing device are used as input for analysis.

The changes of the sequencing workflow introduced by the method of the invention are illustrated in FIG. 6.

The data analysis steps of the invention can be assigned to different general categories and include, for example, combinations of the following steps. Thereby, the Illumina analysis steps are currently technically required and executed by the manufacturer's software. However, requirements may change in the future, thus these steps may be adapted to fulfill potential new requirements. The list of all analysis steps is exemplary and not intended to limit the scope of the invention. The analysis steps may be modified, omitted, or additional steps may be added:

### Illumina Analysis

Cluster identification (usually performed in the first 5-7 cycles of a sequencing run).
Calibration and Quality Filtering (usually performed in the first 25 cycles of a sequencing run)
Base calling (usually performed for each cycle of the sequencing run)

### Data Preprocessing.

Low quality filter; removes reads of average quality not being sufficient for a specific type of analysis
Low complexity filter; removes reads of low complexity that are usually non-informative and might have negative influence on the interpretation of results.
Low quality trimming; trim the sequence information if the average quality behind this position is not sufficient to be included for a specific type of analysis
Adapter trimming; For sequenced DNA molecules being shorter than the specified read length, the adapter oligonucleotide is sequenced at the end of the read and needs to be removed from the sequence information.
(Human) host filter; Removal of sequences originating from the (human) host of the sample.
Background filter; Removal of sequences originating from organisms specified in a background signal database, e.g. to remove contamination specific to a laboratory or sample preparation kit
Any other preprocessing step; Preprocessing steps being necessary for or improving analyses performed in the workflow

### Data Analysis.

Short read alignment; Compare short reads to a database of interest. Such a database can include organisms, biomarkers, specific genes such as resistance genes, etc.
Taxonomic classification; Assign short reads to be related to a specific taxonomic entry included in a taxonomy-based database
Assembly; Reconstruct a full genome using the short-read information, either using a reference sequence (reference-based assembly) or not (de novo assembly)
Variant calling; Detect differences of sequences in the sample compared to known sequences in a database of interest.
Any other method; A suitable method to answer a question of interest.
Quality control; Analysis of different metrics of the data to deliver quality control for a sequencing run, specific to the full run, a specific sample, specific parts of the flow cell, or other dimensions.

### Data Postprocessing

Migration of results; Combining analysis results for single reads and analysis steps to an overall conclusion
Calculation of confidence; Use workflow-specific metrics, learning-based methods and/or artificial intelligence to estimate confidence of results and whether results might change with ongoing sequencing
Estimation of completeness; Use workflow-specific metrics, learning-based methods and/or artificial intelligence to estimate completeness of results, i.e., predict whether additional conclusions are expected to occur with ongoing sequencing.
Summary of results; Automated creation of a result report based on the overall conclusions of the analysis
Visualization of results; Visualization of the analysis results.
Any other postprocessing step; using analysis results of different reads and/or steps to make overall conclusions and facilitate interpretation of results

In embodiments, the method of the invention is (at least partially) computer implemented. The method may use a computer, a computer network or other programmable apparatus, such as a sequencing machine, for carrying out the real-time data analysis of the sequencing data recorded during the SBS process.

In embodiments, a computer, computer network or other programmable apparatus receives and/or exchanges data with the sequencing machine, in real-time, meaning during the sequencing process, wherein sequence data that have just been generated in an ongoing sequence read are directly provided to the computer, computer network or other programmable apparatus with the computer program for data analysis. In embodiments of the method of the invention, when executed by a computer, computer network or other programmable apparatus, the computer program for data analysis can carry out sample specific data analysis of the DNA sequences of the DNA fragments provided in the respective samples, including the steps of
the assignment of sequencing reads to cluster in the flow cell during the initial 3-10 cycles of the sequencing process, preferably based on the detected random sequence of the adapter oligonucleotide,
the assignment of sequencing reads in the flow cell to the corresponding sample of DNA fragments based on the detected sample-specific barcoding-sequence,
data pre- and post-processing steps, and/or
provision of data analysis results during the sequencing process, for example with respect to the presence of one or more specific DNA sequences in the sample.

In another aspect, the invention relates to an apparatus suitable for carrying out the steps of the present invention.

The present invention can be used in many different contexts where fast sequence analysis of multiple samples comprising nucleic acid sequences is useful or desired. For example, the method of the invention can be used for
the diagnosis of a medical condition, such as an infection and related antimicrobial resistances,
determining microbial compositions of a sample,
diagnosis or prognosis of an autoimmune disease, a transplant rejection reaction, a genetic disorder, or cancer;
the detection of a microbial contamination of a sample, such as a food sample (or any other batch process);
tracing the biological, geographical or any other origin of a sample;
the detection of genetically modified organisms;
the identification of plant pathogens;
the general (sample-specific) quality control of a sequencing run;
for the identification of an optimal time point to stop a sequencing run for cost and usage optimization; or
a forensic or hygiene analysis.

Importantly, since the method of the invention enables provision of sequencing results in real time during the sequencing run, the method can be used for example for diagnostic purposes in the context of a point of care analysis. In embodiments, the method of the invention is used for the detection of specific nucleic acid sequences in multiple samples in parallel. In the context of a hospital or other healthcare facility, samples that have been collected from multiple patients can be analyzed efficiently in parallel in a single reaction vessel for the presence of a specific target sequence, such as an antibiotic-resistance cassette. Accordingly, it is possible to rapidly identify patients with a specific condition based on the detected sequence in real-time during the sequence analysis, and the patient can be subsequently subjected to a suitable treatment. For example, in case of detection of an infection with a bacterium comprising a resistance gene for a certain class of antibiotics, an effective antibiotic can be selected for subsequent treatment.

The present invention is useful for any kind of application, where many different samples are analyzed with respect to the presence of certain nucleic acid sequences. It is highly advantageous to methods of the state of the art, since it enables high throughput analysis of samples due to the possibility of highly parallel analysis in the same reaction vessel, while providing results already during the sequencing reaction. In contrast, parallel sequencing analysis so far cannot provide results during the sequencing run but requires subsequent time-consuming analysis.

In another aspect, the invention concerns a kit real-time sequence analysis comprising
a first adapter oligonucleotide for parallel real-time sequencing according to the present invention,
a second adapter oligonucleotide according to the present invention, wherein the second oligonucleotide is optionally hybridized to the first adapter oligonucleotide,
optionally one or more reagents for connecting, e.g. ligating, the adapter oligonucleotides to 5′ ends of DNA fragments comprised in a sample,
and a computer program, preferably stored on a computer readable medium, for real-time analysis of sequencing data generated in a sequencing process using the adapter oligonucleotides.

Preferably, the kit of the invention comprises more than one kind of first and second adapter oligonucleotides of the invention, wherein different kinds of first and second adapter oligos have different barcoding sequences, to enable performing the method for parallel real-time sequence analysis of the present invention. In embodiments, the kit comprises 2, 3, 4, 5, 6, 7, 8, 9, 10, 15, 20, 25, 30, 40, 50, 60, 70, 80, 90, 100, 150, or more different kinds or first and second adapter oligonucleotides with differentiable barcoding sequences.

In embodiments, the kit of the invention comprises disposable material useful for carrying out the method of the invention, such as for example magnetic beads.

In embodiments, the kit of the invention comprises one or more reagents for additional reaction steps that might be necessary for the preparation of a sequencing library, such as reagents for amplification and purification steps.

In embodiments, the kit can comprise one or more reagents that are required for the SBS sequencing process, for example sequencing primers.

Embodiments and features that are disclosed in one aspect of the invention, i.e. the adapter or the method of the invention, also read on the other aspects of the invention. For example, features described with respect to the adapter oligonucleotide of the invention also read on the claimed method for real-time sequence analysis of DNA fragments of the invention and vice versa. The various aspects of the invention are all based on the unifying concept that positioning a barcoding sequence and preferably also a random sequence downstream of the read 1 sequencing primer site enables real time sequence analysis in the context of a sequencing by synthesis process.

### DETAILED DESCRIPTION OF THE INVENTION

All cited documents of the patent and non-patent literature are hereby incorporated by reference in their entirety.

The present invention is directed to an adapter oligonucleotide for parallel real-time sequencing comprising from 5′ to 3′ a first flow cell binding sequence, a read 1 sequencing primer site, characterized in that 3′ (downstream) from the read 1 sequence primer site there is a sample-specific barcoding sequence.

As used herein, an “adapter oligonucleotide” to an oligonucleotide or oligo, which is a nucleic acid molecule, which is a polymer of nucleotides, either deoxyribonucleotides or ribonucleotides (DNA or RNA oligos), of a relative short length, wherein the nucleotides are joined together by a phosphodiester linkage between 5′ and 3′ carbon atoms. Preferably, in the context of the invention, the term oligo refers to a DNA oligo of up to 200 nucleotides length, such as oligos of about 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, 41, 42, 43, 44, 45, 46, 47, 48, 49, 50, 51, 52, 53, 54, 55, 56, 57, 58, 59, 60, 62, 64, 66, 68, 70, 72, 74, 76, 78, 80, 82, 84, 86, 88, 90, 92, 94, 96, 98, 100, 105, 110, 120, 130, 140, 150, 160, 170, 180, 190 nucleotides.

Oligonucleotides are short DNA or RNA molecules, oligomers, that have a wide range of applications in genetic testing, research, and forensics. Commonly made in the laboratory by solid-phase chemical synthesis, these small bits of nucleic acids can be manufactured as single-stranded molecules with any user-specified sequence, and so are vital for artificial gene synthesis, polymerase chain reaction (PCR), DNA sequencing, molecular cloning and as molecular probes. In nature, oligonucleotides are usually found as small RNA molecules that function in the regulation of gene expression (e.g. microRNA), or are degradation intermediates derived from the breakdown of larger nucleic acid molecules. Oligonucleotides are characterized by the sequence of nucleotide residues that usually make up the entire molecule. The length of the oligonucleotide is usually denoted by “-mer”. For example, an oligonucleotide of six nucleotides (nt) is a hexamer, while one of 25 nt would usually be called a “25-mer”. Oligonucleotides readily bind, in a sequence-specific manner, to their respective complementary oligonucleotides, DNA, or RNA to form duplexes or, less often, hybrids of a higher order. This basic property serves as a foundation for the use of oligonucleotides in detecting specific sequences of DNA or RNA. Examples of procedures that use oligonucleotides include DNA microarrays, Southern blots, ASO analysis, fluorescent in situ hybridization (FISH), PCR, and the synthesis of artificial genes.

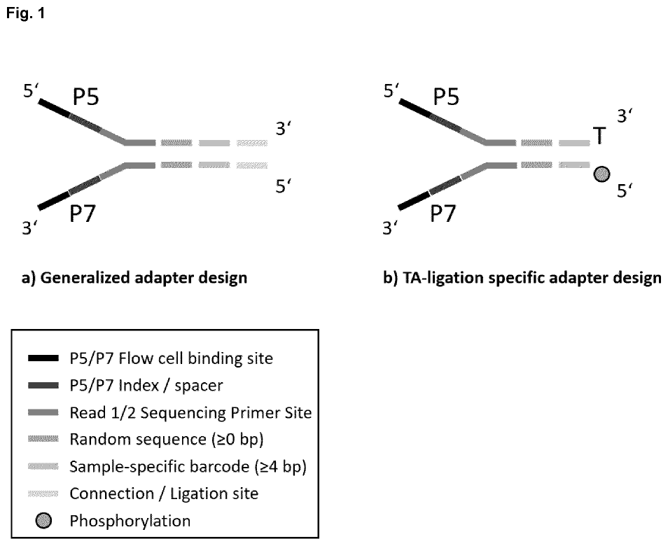

As used herein, the term adapter oligonucleotide can refer to a monomer, meaning a single oligo, or a dimer, meaning two oligos that are connected or bound to each other, for example by hybridization or partial hybridization. Partial hybridization refers to a state where sequence stretches within two oligos or two nucleic acid molecules hybridize, but not the whole sequence of one or both molecules. In the context of the present invention, the terms “adapter” or “adapter oligo(s)” or “adapter oligonucleotide(s)” can refer to a first oligo of the invention, a second oligo of the invention, or a dimer of a first and a second oligo of the invention, which are (partially) hybridized to each other, preferably forming a Y-shaped structure. As used herein, a “Y-shape” refers to a dimer of two oligos which are hybridized to each other on one end and are not hybridized to each other on the other end, so that a schematic representation of the dimer resembles to the letter “Y”, as can be seen in FIG. 1.

The term “hybridization” refers to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.” The term “melting temperature” or “Tₘ” refers to the temperature at which a double stranded nucleic acid melt or dehybridizes. The melting temperature is the temperature at which a population of double-stranded nucleic acid molecules becomes half dissociated into single strands. The equation for calculating the Tₘ of nucleic acids is well known in the art. A simple estimate of the Tₘ value may be calculated by the equation: Tₘ=81.5+0.41 (% G+C), when a nucleic acid is in aqueous solution at 1 M NaCl (See, e.g., Anderson and Young, Quantitative Filter Hybridization, in Nucleic Acid Hybridization (1985)). Other references include more sophisticated computations that take structural as well as sequence characteristics into account for the calculation of Tₘ.

Further, the term “adapter oligo” nucleotide implies that the respective oligo or oligo-dimer is used in a DNA sequencing method as an adapter that is connected to the ends of DNA molecules or DNA fragments, which are to be analyzed/sequenced in an SBS process. As used herein, a DNA fragment or DNA molecule can be a double-stranded (ds) or a single-stranded (ss) DNA molecule. In embodiments of the method of the invention, the adapter oligonucleotides are connected by methods that require ssDNA or that require dsDNA. In case the DNA molecules or fragments are provided in a sample in ds form, ssDNA can be generated by melting the dsDNA at a suitable temperature.

As is well described in the art and known to the skilled person, adapter oligos or adapters are a key component of the next generation sequencing (NGS) workflow. An adapter (or adaptor) is a short, usually chemically synthesized, single-stranded or double-stranded oligonucleotide that can be connected, for example ligated, to the ends of other DNA or RNA molecules. Double stranded adapters can be synthesized to have blunt ends to both terminals or to have sticky end at one end and blunt end at the other. For instance, a double stranded DNA adapter can be used to link the ends of two other DNA molecules (i.e., ends that do not have “sticky ends”, that is complementary protruding single strands by themselves). It may be used to add sticky ends to cDNA allowing it to be ligated into the plasmid much more efficiently. Two adapters could base pair to each other to form dimers.

The adapters and the method of the present invention represent a modification, or an advancement of known adapters and methods used for sequencing of DNA molecules. The invention is based on the commonly used next generation sequencing (NGS) technology the uses a sequencing by synthesis (SBS) process. This process is widely known in the art and has been described extensively, as is known to the skilled person. The most commonly used SBS technology provided by the company Illumina is described in Technology Spotlight: Illumina® Sequencing (Pub. No. 770-2007-002, Current as of 11 Oct. 2010; see also Bentley D R, Balasubramanian S, Swerdlow H P, et al. Accurate Whole Human Genome Sequencing using Reversible Terminator Chemistry. Nature. 2008; 456 (7218): 53-59.).

NGS using SBS is a technique used to determine the series of base pairs in DNA, also known as DNA sequencing. The reversible terminated chemistry concept was invented by Bruno Canard and Simon Sarfati at the Pasteur Institute in Paris and was developed by Shankar Balasubramanian and David Klenerman of Cambridge University, who subsequently founded Solexa, a company later acquired by Illumina. This sequencing method is based on reversible dye-terminators that enable the identification of single nucleotides as they are washed over DNA strands. It can also be used for whole-genome and region sequencing, transcriptome analysis, metagenomics, small RNA discovery, methylation profiling, and genome-wide protein-nucleic acid interaction analysis. The technology works in three basic steps: amplify, sequence, and analyze.

The process begins with provision of DNA, purified DNA or purified DNA fragments. The DNA can get fragmented up into smaller pieces of preferably less than 1000 nucleotides/base pairs and given adapters, potentially barcoding-sequences and other kinds of molecular modifications that act as reference points during amplification, sequencing, and analysis are added. The modified DNA is loaded onto a specialized chip where amplification and sequencing will take place. Along the bottom of the chip are hundreds of thousands or even millions or billions of oligonucleotides (short, synthetic pieces of DNA). They are anchored to the chip and able to grab DNA fragments that have complementary adapter sequences. Once the fragments have attached, cluster generation begins. Cluster generation results in about a thousand copies of each fragment of DNA. Next, primers and modified nucleotides enter the chip and these nucleotides have reversible 3′ blockers that force the polymerase to add on only one nucleotide at a time as well as fluorescent tags. After each round of synthesis, a camera takes a picture of the chip. A computer determines what base was added by the wavelength of the fluorescent tag and records it for every spot on the chip. After each round, non-incorporated molecules are washed away. A chemical deblocking step is then used in the removal of the 3′ terminal blocking group and the dye in a single step. The process may continue until the full DNA molecule is sequenced. With this technology, thousands of places throughout the genome are sequenced at once via massive parallel sequencing.

Accordingly, for Illumina sequencing, as for other sequencing technologies, it is required to provide purified double-stranded DNA fragments with a length of preferably no more than 1000 nucleotides and suitable adapter sequences at both ends. DNA molecules of a sample may be fragmented to have a suitable length. When using tagmentase-based approaches for library preparation, fragmentation and adapter connection can be performed in a single reaction. Therefore, pooling of DNA material from different samples usually occurs after library preparation including connecting the adapters to the ends of the DNA fragments.

The term “dsDNA molecule” refers to a dsDNA composed of two complementary strands of DNA that are bound to each other via base-pairing. Although a dsDNA molecules is composed of two individual DNA molecules, the term as used herein refers to the hybridized complex of two DNA strands.

As explained, the DNA is usually fragmented, and adapters are added that contain segments that act as reference points during amplification, sequencing, and analysis. The modified DNA is loaded onto a flow cell, which is the reaction vessel of the sequencing process, where amplification and sequencing will take place. Some types of flow cells are patterned with nanowells that space out fragments and help with overcrowding. Each nanowell contains oligonucleotides, which are usually fixed with their 5′end on the flow cell surface, so that the 3′ end is free and can interact/hybridize to DNA fragments. These flow cell oligos provide an anchoring point for the adaptors that are linked to the DNA fragments to attach. Once the fragments have attached, a phase called cluster generation begins. This step usually makes about a thousand copies of each fragment of DNA and is done by bridge amplification PCR.

Next, primers (such as a read 1 primer) and modified nucleotides are washed onto the chip, meaning that they are introduced into the flow cell. These nucleotides have a reversible 3′ fluorescent blocker so the DNA polymerase can only add one nucleotide at a time onto the DNA fragment. After each round of synthesis, a camera takes a picture of the chip. A computer determines what base was added by the wavelength of the fluorescent tag and records it for every spot on the chip. After each round, non-incorporated molecules are washed away. A chemical deblocking step is then used to remove the 3′ fluorescent terminal blocking group. The process continues until the full DNA molecule is sequenced. With this technology, thousands of places throughout the genome are sequenced at once via massive parallel sequencing.

The DNA library for sequencing, such as a genomic library of a whole (human) genome, is prepared by isolating the total DNA to be analyzed. After the DNA is purified a DNA library, such as a genomic library, needs to be generated. There are several ways a genomic library can be created, including sonification and tagmentation and others, such as other enzymatic fragmentation methods. With tagmentation, transposases randomly cuts the DNA into sizes between 50 to 500 bp fragments and adds adaptors simultaneously (Clark, David P. (2 Nov. 2018). Molecular biology. Pazdernik, Nanette Jean, McGehee, Michelle R. (Third ed.). London. ISBN 978-O-12-813289-0). A genetic library can also be generated by using sonification to fragment genomic DNA. Sonification fragments DNA into similar sizes using ultrasonic sound waves. Right and left adapters can be attached by T7 DNA Polymerase and T4 DNA ligase after sonification. Strands that fail to have adapters ligated are washed away. Further ways of library preparation and adapter-connection to DNA fragments to be sequenced are known in the art, as described for example by Head et al (“Library construction for next-generation sequencing: Overviews and challenges”, Biotechniques 56 (2): 61-passim. doi:10.2144/000114133).

Classical Illumina sequencing adapters contain three different sequence segments: the sequence complementary to a sequence of the flow cell oligo on the solid support, the barcode sequence (indices), and the binding site for the sequencing primer. Indices are usually six to ten base pairs long and are used during DNA sequence analysis to identify samples. Via a so-called dual index strategy, different combinations of indices allow to distinguish even more different samples than with the use of only a single index sequence. With such strategies, it is generally possible to run hundreds to thousands of samples on a single sequencing run with a sufficiently large high-throughput sequencing device. The general strategy of using specific index sequences to distinguish samples is known as multiplexing. During analysis, which takes place after the sequencing process is completed, the computer will group all reads with the same index together.

Illumina uses a “sequence by synthesis” approach which takes place inside of an acrylamide-coated glass flow cell. The flow cell has oligonucleotides (short nucleotide sequences) coating the bottom of the cell, and they serve as the solid support to hold the DNA strands in place during sequencing. As the fragmented DNA is washed over the flow cell, the appropriate adapter attaches to the complementary solid support. Once attached, a process called cluster generation can begin. The goal is to create hundreds of identical strands of DNA. Some will be the forward strand; the rest, the reverse. This is why right and left adapters (corresponding to the first and second adapters of the invention) are used. Clusters are generated through bridge amplification. DNA polymerase moves along a strand of DNA, creating its complementary strand. The original strand is washed away, leaving only the reverse strand. At the top of the reverse strand there is an adapter sequence. The DNA strand bends and attaches to the oligo that is complementary to the top adapter sequence. Polymerases attach to the reverse strand, and its complementary strand (which is identical to the original) is made. The new double stranded DNA is denatured so that each strand can separately attach to an oligonucleotide sequence anchored to the flow cell. One will be the reverse strand; the other, the forward. This process is called bridge amplification, and it happens for thousands to millions of clusters all over the flow cell at once. In bridge amplification, DNA strands will bend and attach to the solid support many times and each time the DNA polymerase will synthesize a new strand to create a double stranded segment, and that will be denatured so that all of the DNA strands in one area (cluster) are from a single source (clonal amplification). Clonal amplification can be important for quality control purposes. If a strand is found to have an odd sequence, then scientists can check the reverse strand to make sure that it has the complement of the same oddity. The forward and reverse strands can therefore act as checks to guard against artefacts. Because Illumina sequencing uses DNA polymerase, base substitution errors have been observed, especially at the 3′ end. Paired end reads combined with cluster generation can confirm an error took place. The reverse and forward strands should be complementary to each other, all reverse reads should match each other, and all forward reads should match each other. If a read is not similar enough to its counterparts (with which it should be a clone), an error may have occurred.

At the end of clonal amplification, all of the reverse strands are washed off the flow cell, leaving only forward strands. A primer (the so-called read 1 primer) attaches to the forward strands adapter (read 1) primer binding site, and a polymerase adds a fluorescently tagged dNTP to the DNA strand. Only one base is able to be added per round due to the fluorophore acting as a blocking group; however, the blocking group is reversible. Using the four-color chemistry, each of the four bases has a unique emission, and after each round, the machine records which base was added. Once the color is recorded the fluorophore is washed away and another dNTP is washed over the flow cell and the process is repeated. dATPs, dTTPs, dGTPs, and dCTPs are washed over the cell separately so each nucleotide is able to be identified. Once the DNA strand has been read, the strand that was just added is washed away. Then, the index 1 primer attaches, polymerizes the index 1 sequence, which in known sequencing techniques and adapters is located upstream/5′ of the (read 1) primer binding site, and is subsequently washed away. The strand forms a bridge again (after de-blocking the 3′end of the strand), and the 3′ end of the DNA strand attaches to an oligo on the flow cell. The index 2 primer attaches, polymerizes the sequence, and is washed away. A polymerase sequences the complementary strand on top of the arched strand. They separate, and the 3′ end of each strand is blocked. The forward strand is washed away, and the process of sequence by synthesis repeats for the reverse strand.

In this context, the sequencing step starting from the read 1 primer may be referred to as the first sequencing read. The subsequent sequencing reactions, such as the one starting from the index 1 and the index 2 primer, may also be called reads and can be numbered in the order as they occur during the process.

Starting with the launch of the NextSeq and later the MiniSeq, Illumina introduced a new two-color sequencing chemistry. Nucleotides are distinguished by either one of two colors (red or green), no color (“black”) or combining both colors (appearing orange as a mixture between red and green).

The previous description of the SBS process is given for dual index, paired-end sequencing using a sequencing device relying on a four-color chemistry such as the Illumina MiSeq or HiSeq. While the general sequencing process remains the same, there exist other devices relying on a two-color (e.g., Illumina NextSeq, MiniSeq and NovaSeq) or one-color chemistry (Illumina iSeq). These technologies make use of chained fluorescent block groups that are removed one after each other to identify the synthesized nucleotide. Additionally, there are sequencing protocols where only a single read is sequenced (single-end sequencing) or that use only a single index sequence for multiplexing. However, for all technologies and protocols to date, the sample identification is only possible after finishing the sequencing process of the first sequence read (plus all index sequences).

In classical NGS process, the data analysis occurs after the sequencing reaction has been finished. The sequencing occurs for millions of clusters at once, and each cluster has ˜1,000 identical copies of a DNA insert. The sequence data can be analyzed in very different ways, depending on the question to be answered. One of the most popular analysis methods is the assembly of a full genome. This type of analysis is performed by finding fragments with overlapping areas, called contigs, and lining them up. If a reference sequence is known, the contigs can then compared to it for variant identification. This piecemeal process allows scientists to see the complete sequence even though an unfragmented sequence was never run; however, because Illumina read lengths are not very long (the maximum sequence length that can currently be achieved is 2×300 bp in a paired-end sequencing run on an Illumina MiSeq device), it can be a struggle to resolve certain details of the genomic sequence such as short tandem repeat areas. Another approach that is getting more and more popular is metagenomic shotgun sequencing. With this method, a sample from a specific environment is sequenced, such as soil, water, or the blood and other types of samples from a human patient. This approach allows the researcher or clinician to investigate the microbial composition of a sample. Via taxonomic classification approaches, all the sequence reads are assigned to a specific organism. In a clinical setup, for example, such approaches can identify the cause of disease for a patient without the need to perform the time-consuming steps of cultivation, isolation and read assembly. Besides these two general examples of sequence data analysis, there are many other types of analysis that can be applied depending on the question to be answered.

While Illumina sequencing is the current state-of-the-art sequencing method, there are new sequencing methods arising. Two other SBS-based approaches include the SMRT sequencing technology of Pacific Biosciences and the DNBSEQ technology of MGI, a subsidiary of the BGI group. The key parameters and the library preparation of SMRT sequencing is very different to that of Illumina sequencing and allows for much longer, highly accurate reads and also implicitly enables real-time analysis of the sequencing data. On the other hand, the throughput is much lower, more input DNA is required and the price per base pair is much higher than for Illumina sequencing. Because of the high differences in the general sequencing approach and the possibility of real-time analysis, this technology is not relevant in the context of this invention.

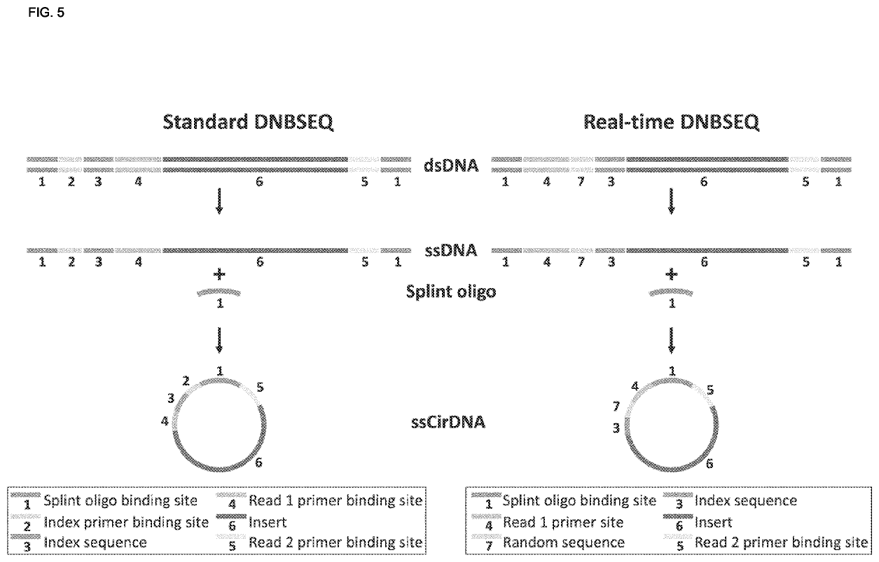

DNBSEQ, in contrast, follows a similar general SBS-approach as Illumina sequencing. The major differences include that the sequencing library contains single-stranded circular DNA molecules. Via circular amplification that is performed even before loading the sample to the flow cell, the complete molecule is amplified to a long single-stranded DNA molecule that consists of a chain of hundreds of copies of the original molecule. On a structural level, this DNA strand forms a ball-shape, which is why these molecules are called nanoballs. In the SBS step, which is performed on a patterned flow cell, the sequencing primer binds to all copies of the sequencing primer binding site that leads to a fluorescent signal that is strong enough to identify the incorporated nucleotides in the SBS approach. As for Illumina sequencing, the standard protocol of DNBSEQ can also not deliver barcode sequences at the beginning of sequencing. In single-end sequencing, the index sequence is sequenced after the first sequence read. In paired-end sequencing, the index sequence is sequenced after the second sequence read. Thus, for both protocols, DNBSEQ provides multiplex information only as the last step of sequencing. This design was presumably chosen for the same reasons as for Illumina sequencing; a sufficient sequencing quality can only be achieved when using the first sequencing cycles for cluster detection and calibration which is not optimal when having low diversity due to index sequences placed at the beginning of sequencing. Therefore, the method of the invention for parallel real-time sequence analysis can be adapted to be also applied with DNBSEQ sequencing technology. The library preparation follows the same general steps as for Illumina sequencing, mainly consisting of fragmentation and adapter binding. The main difference is that after these steps, a circularization step is performed to produce the single-stranded circular structure of the molecule. Therefore, instead of a flow cell binding site, both ends of the double-stranded linear molecule which is present after adapter binding have a region that is complementary to a splint oligo which is used for circularization. When adapting this major difference and some details in the design of the oligonucleotides, the present invention could be suitable to enable real-time analysis for DNBSEQ. An exemplary schematic of the molecular adaptions that would need to be made compared to the standard DNBSEQ protocol is illustrated in FIG. 5, while minor adaptions like introducing a second random sequence and second barcode sequence behind the read 2 primer site might be possible. The software of the invention used for analysis needs to be adapted to support the raw data format of DNBSEQ sequencing devices, and to take technology-specific properties of the data into account.

A third alternative sequencing technology, Oxford Nanopore sequencing, relies on a completely different technology by monitoring changes in an electrical current as nucleic acids are passed through a protein nanopore. While this technology can produce reads of much higher length and implicitly allows for real-time analysis of the data, it has a much lower throughput, higher error rates and has higher costs per base pair than Illumina sequencing. As the underlying biochemistry is completely different to that of SBS-based approaches, this technology is not relevant in the context of this invention.

The method and adapter oligonucleotides of the present invention have been modified in comparison to the known Illumina process to enable parallel real-time sequence analysis during the sequencing run. Importantly, the sequence segments comprised by the adapter oligonucleotides of the invention have been modified. Importantly, in the first adapter oligo, a barcoding sequence is now located downstream (3′) of the read 1 primer site so it is sequenced and detected in the first sequencing run starting from the read 1 primer. Correspondingly, in the context of the second oligo, which is preferably attached to the 5′ end of a provided DNA fragment in the context of the method of the invention, the read 2 sequencing primer site is located 3′ of the barcoding sequence, which is complementary to the barcoding sequence of the first adapter oligo. Accordingly, after connecting the first oligo to the 5′ end of a ssDNA fragment and the second oligo to the 3′ end of the same ssDNA fragment, the barcoding sequence are localized internally from the read 1 and 2 primer binding sites, respectively, meaning that the primer binding sites, are located further towards the respective end of the DNA fragment.

As used herein, a primer site refers to a sequence segment of the adapter oligonucleotide, that enables hybridization of a sequencing primer (also referred to as a “read primer”) during the SBS process.

Furthermore, the innovative arrangement of the sequence segments of the adapter oligos of the invention allows a modification of the steps of the SBS process in comparison the classical approach. Importantly, the method of the invention does not require the previously obligatory index 1 and index 2 read steps for enabling multiplexing/parallel sequencing of DNA fragments form multiple samples in the same flow cell, since the barcoding sequences of the adapter oligo are read in the context of the sequence reads starting from the read 1 and the read 2 primers.

As used herein, a first flow cell binding sequence is a sequence that is preferably located at the 5′ end of the first adapter oligonucleotide of the invention and that enables hybridization to a sequence of a first flow cell oligo. Accordingly, a DNA fragment whose 5′ end has been connected to the 3′ end of a first adapter oligonucleotide, can bind to or hybridize to the first flow cell oligo via the first flow cell binding sequence.

Similarly, the second flow cell binding sequence is located preferably at the 3′ end of the second adapter oligonucleotide of the invention and enables hybridization to a sequence of a second flow cell oligo during the SBS process. In embodiments of the method of the invention, the second adapter oligo is connected with its 5′ end to the 3′ end of a provided DNA fragment, so that the second flow cell binding sequence of the second adapter is located at the 3′ end of the resulting fragment. During the SBS process, a complementary strand to this (forward) DNA fragment is generated, whose 5′ end is complementary to the second flow cell binding sequence and is practically complementary to a sequence of the second flow cell oligo and enables hybridization, for example during bridge amplification.

The length of the flow cell binding sequences is variable and can be adjusted by the skilled person according to the specific application. Preferably, a flow cell binding sequence is about 5-50 nucleotides long, such as 6, 7, 8, 9, 10, 11, 12, 13, 14, 15, 16, 17, 18, 19, 20, 21, 22, 23, 24, 25, 26, 27, 28, 29, 30, 31, 32, 33, 35, 36, 37, 38, 39, 40, 41, 42, 43, 44, 45, 46, 47, 48, 49 nucleotides. In the context of the invention, known flow cell binding sequences, such as the P5 and P7 sequences disclosed in the examples below, can be used. This is advantageous, since these sequences make the method of the invention compatible with standard equipment. Particularly preferred length of flow cell binding sequences are about 20-30 nucleotides long.

The method of the present invention may be used for parallel real-time genome sequence analysis. The term “genome” as used herein is defined as the collective gene set carried by an individual, cell, or organelle. The term “genomic DNA” as used herein is defined as DNA material comprising the partial or full collective gene set carried by an individual, cell, or organelle. In embodiments, the method may be used for metagenomic analysis. The term “metagenomic” as used herein is defined as the full or partial set of DNA directly obtained from an environmental sample, for example soil, water, blood, respiratory samples, swaps, and others. In embodiments, the method may be used for transcriptome analysis. The term “transcriptome” as used herein is defined as the collective RNA set expressed within a cell, which can be reverse transcribed to cDNA for sequencing analysis. In embodiments, the method may be used for metatranscriptomic analysis. The term “metatranscriptomic” as used herein is defined as the full or partial set of RNA expressed within any cell of an environmental sample, which can be reserve transcribed to cDNA for sequence analysis. In embodiments, the method may be used for all types of samples that can be sequenced with the specified SBS approach. As used herein, the term “nucleoside” refers to a molecule having a purine or pyrimidine base covalently linked to a ribose or deoxyribose sugar. Exemplary nucleosides include adenosine, guanosine, cytidine, uridine and thymidine. Additional exemplary nucleosides include inosine, 1-methyl inosine, pseudouridine, 5,6-dihydrouridine, ribothymidine, 2N-methylguanosine and 2′2N, N-dimethylguanosine (also referred to as “rare” nucleosides). The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides, either deoxyribonucleotides or ribonucleotides, of any length joined together by a phosphodiester linkage between 5′ and 3′ carbon atoms. Polynucleotides can have any three-dimensional structure and can perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. The terms oligonucleotide, polynucleotide and nucleic acid molecule may refer to both double- and single-stranded molecules. Unless otherwise specified or required, any embodiment of this invention that comprises a polynucleotide or nucleic acid reads on both the double-stranded form and each of two complementary single-stranded forms known or predicted to make up the double-stranded form, as is understood by the skilled person in the context of the respective disclosure. A polynucleotide is composed of a specific sequence of four nucleotide bases: adenine (A); cytosine (C); guanine (G); thymine (T); and uracil (U) for thymine when the polynucleotide is RNA. Thus, the term polynucleotide sequence is the alphabetical representation of a polynucleotide molecule. This alphabetical representation can be input into databases in a computer having a central processing unit and used for bioinformatics applications such as functional genomics and homology searching. The terms “RNA,” “RNA molecule” and “ribonucleic acid molecule” refer to a polymer of ribonucleotides. The terms “DNA,” “DNA molecule” and “deoxyribonucleic acid molecule” refer to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double stranded, i.e., dsRNA and dsDNA, respectively). “mRNA” or “messenger RNA” is single-stranded RNA that specifies the amino acid sequence of one or more polypeptide chains. This information is translated during protein synthesis when ribosomes bind to the mRNA.

In embodiments, the adapter oligonucleotides of the invention can comprise nucleotide analogs, altered nucleotides and modified nucleotides. The terms “nucleotide analog,” “altered nucleotide” and “modified nucleotide” refer to a non-standard nucleotide, including non-naturally occurring ribonucleotides or deoxyribonucleotides. In certain exemplary embodiments, nucleotide analogs are modified at any position so as to alter certain chemical properties of the nucleotide yet retain the ability of the nucleotide analog to perform its intended function. Possible modification are labels, such as fluorescent labels. Examples of positions of the nucleotide which may be derivitized include the 5 position, e.g., 5-(2-amino) propyl uridine, 5-bromo uridine, 5-propyne uridine, 5-propenyl uridine, etc.; the 6 position, e.g., 6-(2-amino) propyl uridine; the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O- and N-modified (e.g., alkylated, e.g., N6-methyl adenosine, or as otherwise known in the art) nucleotides; and other heterocyclically modified nucleotide analogs such as those described in Herdewijn, Antisense Nucleic Acid Drug Dev., 2000 Aug. 10 (4): 297-310. Nucleotide analogs may also comprise modifications to the sugar portion of the nucleotides. For example the 2′ OH-group may be replaced by a group selected from H, OR, R, F, Cl, Br, I, SH, SR, NH₂, NHR, NR₂, COOR, or OR, wherein R is substituted or unsubstituted C1-C6 alkyl, alkenyl, alkynyl, aryl, etc. Other possible modifications include those described in U.S. Pat. Nos. 5,858,988, and 6,291,438.

As used herein, the terms “complementary” and “complementarity” are used in reference to nucleotide sequences related by the base-pairing rules. For example, the sequence 5′-AGT-3′ is complementary to the sequence 5′-ACT-3′. Complementarity can be partial or total. Partial complementarity occurs when one or more nucleic acid bases is not matched according to the base pairing rules. Total or complete complementarity between nucleic acids occurs when each and every nucleic acid base is matched with another base under the base pairing rules. The degree of complementarity between nucleic acid strands has significant effects on the efficiency and strength of hybridization between nucleic acid strands. The term “homology” when used in relation to nucleic acids refers to a degree of complementarity. There may be partial homology (i.e., partial identity) or complete homology (i.e., complete identity). A partially complementary sequence is one that at least partially inhibits a completely complementary sequence from hybridizing to a target nucleic acid and is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe (i.e., an oligonucleotide which is capable of hybridizing to another oligonucleotide of interest) will compete for and inhibit the binding (i.e., the hybridization) of a completely homologous sequence to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target which lacks even a partial degree of complementarity (e.g., less than about 30% identity); in the absence of non-specific binding the probe will not hybridize to the second non-complementary target. When used in reference to a double-stranded nucleic acid sequence such as a cDNA or genomic clone, the term “substantially homologous” refers to any probe or primer or oligonucleotide which can hybridize to either or both strands of the double-stranded nucleic acid sequence under conditions of low stringency. When used in reference to a single-stranded nucleic acid sequence, the term “substantially homologous” refers to any probe which can hybridize to the single-stranded nucleic acid sequence under conditions of low stringency.

The following terms are used to describe the sequence relationships between two or more polynucleotides: “reference sequence,” “sequence identity,” “percentage of sequence identity” and “substantial identity”. A “reference sequence” is a defined sequence used as a basis for a sequence comparison; a reference sequence may be a subset of a larger sequence, for example, as a segment of a full-length cDNA sequence given in a sequence listing or may comprise a complete gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window”, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman (Smith and Waterman (1981) Adv. Appl. Math. 2:482) by the homology alignment algorithm of Needleman and Wunsch (J. Mol. Biol. 48:443 (1970)), by the search for similarity method of Pearson and Lipman (Proc. Natl. Acad Sci. USA 85:2444 (1988)]), by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected. The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a segment of the full-length sequences of the compositions claimed in the present invention.

The term DNA fragment or DNA molecule, as used in the context of the method of the invention, refers to a DNA that is comprised in a sample or derived from nucleic acids molecules comprised in a sample by processing. Nucleic acids that can be processed to provide the DNA fragments to be analyzed in the context of the present invention may be DNA, RNA, or DNA-RNA chimeras, and they may be obtained from any useful source, such as, for example, a human sample. The nucleic acids provided in a sample or specimen can be processed to be converted to DNA molecules or DNA fragments to be analyzed (sequenced) in the method of the invention. In specific embodiments, a double stranded DNA molecule is further defined as comprising a genome, such as, for example, one obtained from a sample from a human. The sample may be any sample from a human, such as blood, serum, plasma, cerebrospinal fluid, cheek scrapings, nipple aspirate, biopsy, semen (which may be referred to as ejaculate), urine, feces, hair follicle, saliva, sweat, immunoprecipitated or physically isolated chromatin, and so forth. In specific embodiments, the sample comprises a single cell. In embodiments, a sample comprises a tissue sample or multiple cells. In particular embodiments, the sequenced DNA fragment resulting from one or more nucleic acid molecule from a sample provides diagnostic or prognostic information. For example, the prepared nucleic acid molecule from the sample may provide genomic copy number and/or sequence information, allelic variation information, cancer diagnosis, prenatal diagnosis, paternity information, disease diagnosis, detection, monitoring, and/or treatment information, sequence information, and so forth.

As used herein, the term “primer” generally includes an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process are determined by the sequence of the template polynucleotide. Usually primers are extended by a DNA polymerase. Primers usually have a length in the range of between 3 to 36 nucleotides, also 5 to 24 nucleotides, also from 14 to 36 nucleotides. Primers within the scope of the invention include orthogonal primers, amplification primers, constructions primers and the like. Pairs of primers can flank a sequence of interest or a set of sequences of interest. Primers and probes can be degenerate or quasi-degenerate in sequence. Primers within the scope of the present invention bind adjacent to a target sequence. A “primer” may be considered a short polynucleotide, generally with a free 3′-OH group that binds to a target or template potentially present in a sample of interest by hybridizing with the target, and thereafter promoting polymerization of a polynucleotide complementary to the target. Primers of the instant invention are comprised of nucleotides ranging from 10 to 30 nucleotides. In one aspect, the primer is at least 10 nucleotides, or alternatively, at least 11 nucleotides, or alternatively, at least 12 nucleotides, or alternatively, at least 13 nucleotides, or alternatively, at least 14 nucleotides, or alternatively, at least 15 nucleotides, or alternatively, at least 16 nucleotides, or alternatively, at least 16 nucleotides, or alternatively, at least 17 nucleotides, or alternatively, at least 18 nucleotides, or alternatively, at least 19 nucleotides, or alternatively, at least 20 nucleotides, or alternatively, at least 21 nucleotides, or alternatively, at least 22 nucleotides, or alternatively, at least 23 nucleotides, or alternatively, at least 24 nucleotides, or alternatively, at least 25 nucleotides, or alternatively, at least 26 nucleotides, or alternatively, at least 27 nucleotides, or alternatively, at least 28 nucleotides, or alternatively, at least 29 nucleotides, or alternatively, at least 30 nucleotides, or alternatively at least 50 nucleotides, or alternatively at least 75 nucleotides or alternatively at least 100 nucleotides.

The processes of Library preparation from nucleic acids provided in a sample to be analyzed can comprise DNA amplification steps using methods known to those of skill in the art. In certain aspects, amplification is achieved using PCR. The term “polymerase chain reaction” (“PCR”) of Mullis (U.S. Pat. Nos. 4,683,195, 4,683,202, and 4,965,188) refers to a method for increasing the concentration of a segment of a target sequence in a mixture of nucleic acid sequences without cloning or purification. This process for amplifying the target sequence consists of introducing a large excess of two oligonucleotide primers to the nucleic acid sequence mixture containing the desired target sequence, followed by a precise sequence of thermal cycling in the presence of a polymerase (e.g., DNA polymerase). The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle;” there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.” With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications. Methods and kits for performing PCR are well known in the art. PCR is a reaction in which replicate copies are made of a target polynucleotide using a pair of primers or a set of primers consisting of an upstream and a downstream primer, and a catalyst of polymerization, such as a DNA polymerase, and typically a thermally-stable polymerase enzyme. Methods for PCR are well known in the art, and taught, for example in MacPherson et al. (1991) PCR 1: A Practical Approach (IRL Press at Oxford University Press). All processes of producing replicate copies of a polynucleotide, such as PCR or gene cloning, are collectively referred to herein as replication. A primer can also be used as a probe in hybridization reactions, such as Southern or Northern blot analyses. The expression “amplification” or “amplifying” refers to a process by which extra or multiple copies of a particular polynucleotide are formed. Amplification includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., “PCR protocols: a guide to method and applications” Academic Press, Incorporated (1990) (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified. Reagents and hardware for conducting amplification reaction are commercially available. Primers useful to amplify sequences from a particular gene region are preferably complementary to, and hybridize specifically to sequences in the target region or in its flanking regions and can he prepared using the polynucleotide sequences provided herein. Nucleic acid sequences generated by amplification can be sequenced directly. When hybridization occurs in an antiparallel configuration between two single-stranded polynucleotides, the reaction is called “annealing” and those polynucleotides are described as “complementary”. A double-stranded polynucleotide can be complementary or homologous to another polynucleotide, if hybridization can occur between one of the strands of the first polynucleotide and the second. Complementarity or homology (the degree that one polynucleotide is complementary with another) is quantifiable in terms of the proportion of bases in opposing strands that are expected to form hydrogen bonding with each other, according to generally accepted base-pairing rules.

The terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences. Such molecules are comprised by the term DNA fragment of a sample. The term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.). Amplification methods include PCR methods known to those of skill in the art and also include rolling circle amplification (Blanco et al., J. Biol. Chem., 264, 8935-8940, 1989), hyperbranched rolling circle amplification (Lizard et al., Nat. Genetics, 19, 225-232, 1998), and loop-mediated isothermal amplification (Notomi et al., Nuc. Acids Res., 28, e63, 2000) each of which are hereby incorporated by reference in their entireties.

“Identity,” “homology” or “similarity” are used interchangeably and refer to the sequence similarity between two nucleic acid molecules. Identity can be determined by comparing a position in each sequence which can be aligned for purposes of comparison. When a position in the compared sequence is occupied by the same base or amino acid, then the molecules are homologous at that position. A degree of identity between sequences is a function of the number of matching or identical positions shared by the sequences. An unrelated or nonhomologous sequence shares less than 40% identity, or alternatively less than 25% identity, with one of the sequences of the present invention. A polynucleotide has a certain percentage (for example, 60%, 65%, 70%, 75%, 80%, 85%, 90%, 95%, 98% or 99%) of “sequence identity” to another sequence means that, when aligned, that percentage of bases are the same in comparing the two sequences. This alignment and the percent sequence identity or homology can be determined using software programs known in the art, for example those described in Ausubel et al., Current Protocols in Molecular Biology, John Wiley & Sons, New York, N.Y., (1993). Preferably, default parameters are used for alignment. One alignment program is BLAST, using default parameters. In particular, programs are BLASTN and BLASTP, using the following default parameters: Genetic code=standard; filter=none; strand=both; cutoff-60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the National Center for Biotechnology Information.

The practice of certain embodiments or features of certain embodiments may employ, unless otherwise indicated, conventional techniques of molecular biology, microbiology, recombinant DNA, and so forth which are within ordinary skill in the art. Such techniques are explained fully in the literature. See e.g., Sambrook, Fritsch, and Maniatis, MOLECULAR CLONING: A LABORATORY MANUAL, Second Edition (1989), OLIGONUCLEOTIDE SYNTHESIS (M. J. Gait Ed., 1984), ANIMAL CELL CULTURE (R. I. Freshney, Ed., 1987), the series METHODS IN ENZYMOLOGY (Academic Press, Inc.); GENE TRANSFER VECTORS FOR MAMMALIAN CELLS (J. M. Miller and M. P. Calos eds. 1987), HANDBOOK OF EXPERIMENTAL IMMUNOLOGY, (D. M. Weir and C. C. Blackwell, Eds.), CURRENT PROTOCOLS IN MOLECULAR BIOLOGY (F. M. Ausubel, R. Brent, R. E. Kingston, D. D. Moore, J. G. Siedman, J. A. Smith, and K. Struhl, eds., 1987), CURRENT PROTOCOLS IN IMMUNOLOGY (J. E. coligan, A. M. Kruisbeek, D. H. Margulies, E. M. Shevach and W. Strober, eds., 1991); ANNUAL REVIEW OF IMMUNOLOGY; as well as monographs in journals such as ADVANCES IN IMMUNOLOGY.

The skilled person is able to identify on the basis of the listed publications and updated editions thereof suitable techniques to be used in the context of the invention.

Terms and symbols of nucleic acid chemistry, biochemistry, genetics, and molecular biology used herein follow those of standard treatises and texts in the field, e.g., Komberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

As used herein, “diagnosis” in the context of the present invention relates to the recognition and (early) detection of a clinical condition of a subject linked to a disease, for example an infectious disease. Also, the assessment of the severity of a condition, such as for example an infectious disease, may be encompassed by the term “diagnosis”.

“Prognosis” relates to the prediction of an outcome or a specific risk for a subject based on a disease, such as an infectious disease. This may also include an estimation of the chance of recovery or the chance of an adverse outcome for said subject.

As used herein, the “patient” or “subject” may be a vertebrate. In the context of the present invention, the term “subject” includes both humans and animals, particularly mammals, and other organisms.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

Thus, the terms “comprising”/“including”/“having” mean that any further component (or likewise features, integers, steps and the like) can/may be present. The term “consisting of” means that no further component (or likewise features, integers, steps and the like) is present.

The term “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

Thus, the term “consisting essentially of” means those specific further components (or likewise features, integers, steps and the like) can be present, namely those not materially affecting the essential characteristics of the composition, device or method. In other words, the term “consisting essentially of” (which can be interchangeably used herein with the term “comprising substantially”), allows the presence of other components in the composition, device or method in addition to the mandatory components (or likewise features, integers, steps and the like), provided that the essential characteristics of the device or method are not materially affected by the presence of other components.

The term “method” refers to manners, means, techniques and procedures for accomplishing a given task including, but not limited to, those manners, means, techniques and procedures either known to, or readily developed from known manners, means, techniques and procedures by practitioners of the chemical, biological and biophysical arts.

The instant disclosure also includes kits, packages and multi-container units containing the herein described reagents for carrying out the method of the invention.

### FIGURES

The invention is further described by the following figures. These are not intended to limit the scope of the invention but represent preferred embodiments of aspects of the invention provided for greater illustration of the invention described herein.

### BRIEF DESCRIPTION OF THE FIGURES

FIG. 1: FIG. 1 shows the schematic design of the proposed sequencing adapter.

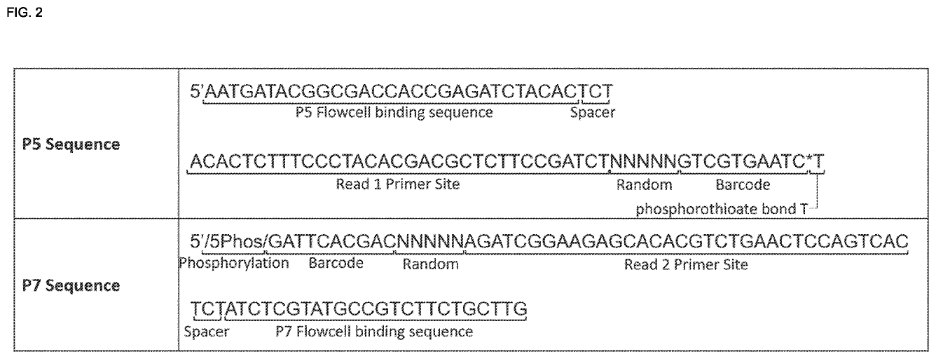

FIG. 2: Example sequence for TA-ligation specific adapters with 5 bp random sequence and 10 bp barcode sequence. Shown is an example of a first adapter oligo with the Sequence SEQ ID NO: 1; 5′AATGATACGGCGACCACCGAGATCTACACTCTACACTCTTTCCCTACACGACGCTC TTCCGATCTNNNNNGTCGTGAATC*T.

FIG. 3: Schematic illustration of the sequencing order of a standard Illumina sequencing approach compared to the parallel real-time sequencing method of the invention.

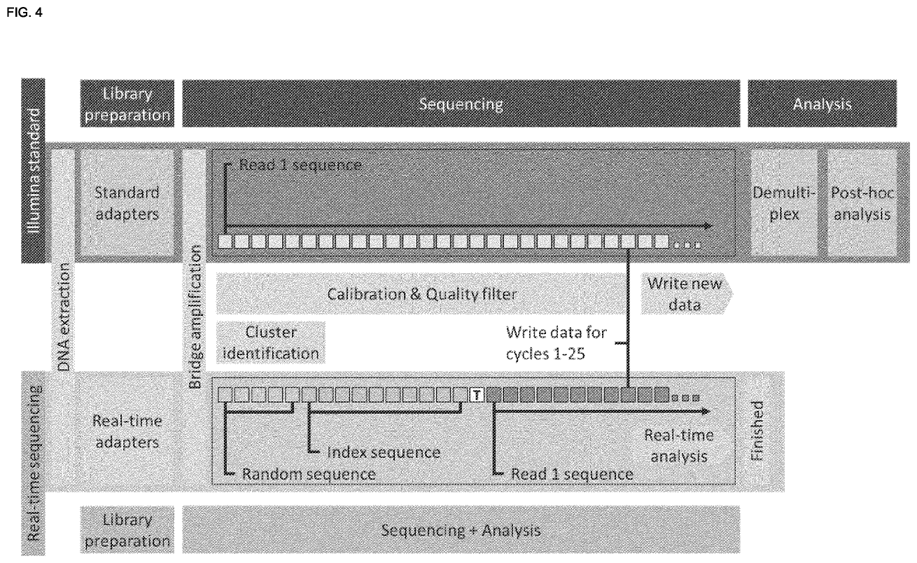

FIG. 4: Schematic illustration of the full sequencing workflow of a standard Illumina sequencing approach compared to the parallel real-time sequencing method of the invention.

FIG. 5: Schematic illustration of a proposed adaption of the sequencing library design to apply the invention with the DNBSEQ sequencing technology of MGI.

FIG. 6: Comparison of a known sequencing workflow as performed by Illumina and a preferred workflow of the invention.

### DETAILED DESCRIPTION OF THE FIGURES

FIG. 1: a) Generalized adapter design for arbitrary types of connection methods of adapter and sequence. b) Adapter design used for TA-ligation as connection method.

FIG. 2: The first row shows an exemplary first P5 sequence adapter oligonucleotide (SEQ ID NO: 1), the second row shows the corresponding second P7 sequence adapter oligonucleotide (SEQ ID NO: 2). Both sequences can be separately synthesized and ligated to form the Y-shape adapters shown in FIG. 1.

FIG. 3: The upper part shows a comparison of standard Illumina sequencing to the parallel real-time sequencing approach of the invention in a paired-end sequencing protocol. Index 2 in Illumina standard sequencing is optional, and Index 1 and Index 2 in parallel real-time sequencing are optional. Parallel real-time sequencing uses the specified real-time index for multiplexing, which originates from the barcode sequence of the oligonucleotide of this invention. The second part shows a comparison of Illumina standard sequencing with the parallel real-time sequencing approach of the invention in a single-end sequencing protocol. Index 1 is optional for parallel real-time sequencing which uses the specified real-time index for multiplexing, which originates from the barcode sequence of the oligonucleotide of this invention. In both parts of the figure, the “ID” tag highlights the time point when the assignment of a read to a specific sample, and therefore a sample-specific analysis, is possible with both protocols.

FIG. 4: The upper (dark) box shows the process of a standard Illumina sequencing procedure. The lower (light) box shows the same process for the parallel real-time sequencing approach of this invention. Identical steps for both approaches are illustrated as long boxes covering the area of both methods. The compared sequencing process is divided into library preparation, sequencing and analysis. In the sequencing part, relevant behavior of the Illumina sequencing device is displayed in the area between both methods. It highlights that the random sequence of our invention is used for cluster identification and ensures sufficient diversity for this step. The analysis step of the process is omitted in parallel real-time sequencing, as the analysis is finished immediately after the sequencing process has finished, while analysis can only start at this time point for standard Illumina sequencing protocols.

FIG. 5: The left side shows the design of the double-stranded DNA molecule after fragmentation and adapter binding. Compared to Illumina sequencing, the most noticeable difference is the replacement of a flow cell binding site by a splint oligo binding site for circularization. The right side shows a proposed adaption of the standard DNBSEQ sequencing library design to apply the parallel real-time sequencing approach of this invention. Minor changes might be applied for specific applications, such as an additional integration of a second random sequence and/or a second index sequence between the insert and read 2 primer binding site.

FIG. 6: The left side shows a standard Illumina sequencing workflow for paired-end sequencing. The base call files of all sequencing segments, including index 1 and index 2, are collected for demultiplexing and file conversion at the end of the sequencing run. Cluster identification and calibration are performed during the first 25 cycles of read 1 of the sequenced DNA molecule/fragment. Demultiplexing is performed by the software delivered by the manufacturer. Data preprocessing, analysis and postprocessing can only be done after demultiplexing and file conversion after the sequencing run finished. Results are available at the end of the full workflow. The right side shows a preferred embodiments of the parallel real-time sequencing workflow of this invention. Compared to standard Illumina sequencing, specialized real-time adapter oligonucleotides are used during library preparation. Cluster identification is performed with the random sequence of the adapter oligonucleotide. Demultiplexing is performed using the sample-specific barcodes of the adapter oligonucleotide after the first base calls were written by the sequencing device (usually after cycle 25). Afterwards, new sequence information is analyzed in real-time when the sequencing device is still running. This continuous analysis includes a novel parallelized concept of data preprocessing, data analysis and data postprocessing. In doing so, real-time results are available still during the sequencing device is running. The separate SBS processes for index 1 and index 2 are not required due to the sample-specific barcode integrated in the real-time oligonucleotide and being sequenced after the random sequence in read 1.

### EXAMPLES

The invention is further described by the following examples. These are not intended to limit the scope of the invention but represent preferred embodiments of aspects of the invention provided for greater illustration of the invention described herein.

### A. General Description of the Inventive Approach

The novel live sequencing method described herein is based on the Illumina sequencing technology and combines a new adapter design for real-time sample assignment with a live data analysis approach.

As previously described, the major problem of real-time sequencing with Illumina sequencing devices is the order of sequenced read segments. When using multiplexing, i.e. sequencing multiple samples in a single run that can be identified via specific barcode sequences, the barcode used for sample assignment is sequenced at the end (single-end) or in the middle (paired-end) of the sequencing run:

Single end sequencing order scheme:
1. Read 1 (50-300 bp)
2. Barcode 1 (6-12 bp)

Paired end sequencing order scheme:
1. Read 1 (50-300 bp)
2. Barcode 1 (6-12 bp)
3. Barcode 2 (6-12 bp)
4. Read 2 (50-300 bp)

When following this standard Illumina protocol, the assignment of a sequence to the correct sample is not possible for the first part of the sequence as the barcode is not yet available at this time point.

As a solution, we propose the use of novel live sequencing adapters that extend the Illumina sequencing adapters by
1. A random sequence at the beginning of the read.
2. An inline-barcode for sample identification.
3. A connection site for the library preparation method used.

A simple reordering of the segments to place the barcodes at the beginning of the sequencing procedure is not easily possible, as Illumina sequencing requires high diversity at the beginning of sequencing to be able to correctly detect the molecular clusters of each sequence.

Additionally, the Illumina software does not allow for placing a barcode at the beginning of the sequence due to technical limitations.

### B. Innovative Adapter Components

The first part of the adapter extension, which is the random sequence, artificially introduces a high sequence diversity at the beginning of sequencing process. The length of the random sequence can in principle be varied; based on the official documentation of Illumina sequencing devices stating that 4-7 bp of high diversity sequence are required to ensure successful cluster detection, we successfully tested the new adapter design on an Illumina MiSeq device with a random sequence of length 5 bp.

The second part of the adapter extension, the inline-barcode, is used for the assignment of the read to a specific sample. The length of the barcode depends on the number of samples that needs to be sequenced. We successfully tested the adapter design with a barcode of length 10 bp.

When using TA-ligation to connect the adapter to the sequence, an additional nucleotide thymine will be present behind the barcode. This artifact must be considered when analyzing the data. In principle, other ligation protocols could be used for connecting the adapter to a sequence (e.g., blunt end ligation). In the following, an example sequence using a 5 bp random segment, 10 bp barcode for TA-ligation protocols is shown:

First adapter oligonucleotide comprising at the 5′ end a P5 Sequence (which is an example first flow cell binding sequence):

(SEQ ID NO: 1)
5′AATGATACGGCGACCACCGAGATCTACACTCTACACTCTTTCCCTAC
ACGACGCTCTTCCGATCTNNNNNGTCGTGAATC*T

The first adapter oligonucleotide of this example consists from 5′ to 3′ of a P5 sequence (underlined), which is an example of a first flow cell binding sequence, followed by a three nucleotide spacer (bold) and the read 1 primer site (italics underlined), a 5 nucleotide random sequence (bold, N can be any of A, T, C or G), the sample-specific barcode (bold underlined) and a T which is connected with a phosphorothioate bond (*) representing the connection site required for TA-ligation, representing a preferred technique for adapter connection to the DNA fragment to be sequenced.

Second adapter oligonucleotide that can be hybridized to the first adapter oligonucleotide described above to provide a partially double-stranded (Y-shaped) adapter comprising at the 3′ end a P7 Sequence (which is an example first flow cell binding sequence):

(SEQ ID NO: 2)
5′/5Phos/GATTCACGACNNNNNAGATCGGAAGAGCACACGTCTGAAC
TCCAGTCACTCTATCTCGTATGCCGTCTTCTGCTTG

The second adapter of oligonucleotide of this example consists from 5′ to 3′ of a phosphorylated 5′-end (/5Phos/) representing a connection site, a sequence complementary to the sample-specific barcoding sequence of the corresponding first adapter oligonucleotide shown above (bold underlined), a 5 nucleotide random sequence which is complementary to the random sequence of the corresponding first adapter oligonucleotide shown above (bold, N can be any of A, T, C or G), the read 2 primer site (italics underlined), a three nucleotide spacer (bold), and a P7 sequence (underlined), which is an example of a second flow cell binding sequence commonly used in the Illumina SBS process.

At specific locations in the adapter design, the sequence can be extended with additional sequences. For example, the spacer region can be omitted, exchanged or extended to one or both sides. For example, the following sequence also forms a valid and functional first adapter oligonucleotide of this invention:

(SEQ ID NO: 31)
5′AATGATACGGCGACCACCGAGATCTACACTCTACACTCTACACTCTT
TCCCTACACGACGCTCTTCCGATCTNNNNNGTCGTGAATC*T

In this variant of the first adapter oligonucleotide, the nucleotide spacer (bold, underlined) is extended with seven additional nucleotides.

The adapters need to be connected to both ends of the fragmented DNA sequences. While the proposed approach using the exemplary adapter oligonucleotides shown herein focuses on TA ligation, it is also possible to use alternative approaches such as a specialized tagmentation reaction to achieve the construction of similar molecules.

The novel adapter design provides a unique combination of advantages that could not be achieved before with other published adapter designs:
There is no limitation in the number and combination of samples and their corresponding barcodes.
High sequence diversity is guaranteed for the first 4-7 cycles that are used for cluster detection, even when sequencing a single sample.
Sample assignment is possible at the beginning of sequencing thanks to front inline barcodes. This enables parallel real-time analysis of multiple samples.
Standard Illumina sequencing primers can be used for sequencing.
The adapters can be used for library preparation protocols with and without (PCR) amplification step.

### C. Software and Analysis Components

In order to utilize the full spectrum of advantages the adapter design offers we combined the application with a specific software.

In this analysis approach, the analysis software considers the new adapter design and assign reads to their corresponding samples by using the inline barcode at the very beginning of the sequencing procedure.

The random adapter sequence that is placed even before the barcode sequence can potentially be used as a variation of unique molecular identifiers during the data analysis. This is particularly useful if an amplification step is included during library preparation.

In combination with the newly developed adapters, it is now possible providing
Sample-specific results already at the beginning of the sequencing (real-time analysis),
Sample-specific quality control for early detection of faulty or low-quality samples (real-time sample-specific quality monitoring),
Include necessary data pre- and post-processing steps and different types of analysis using a novel strategy that allows the base-by-base coupling of different algorithms (real-time data processing), and
Decision making approaches based on artificial intelligence, deep learning, statistical models and hybrid strategies to determine the earliest possible time point when intermediary results can be reliably reported that are not expected to depart from the results at the end of the sequencing run.

The combination of the new adapters and the base-by-base coupling of algorithms with a building blocks system allows delivering high-quality real-time analysis results for nearly all use cases. It can be used to assign analysis results to a specific sample from the beginning of the sequencing process. This innovation allows to significantly reduce the turnaround time from sample arrival to analysis results output.

### D. Parallel Real-Time Sequence Analysis for Pathogen Detection in a Clinical Sample

As an example, the live sequencing approach was used for the detection of pathogens in ten clinical respiratory samples, five of them originating from patients with cystic fibrosis.

The DNA of the samples was extracted using the QIAamp DNA Microbiome Kit (Qiagen GmbH). The library preparation was done with the Lotus DNA Library Prep Kit (Integrated DNA Technologies, Inc.). The live sequencing adapters were synthesized by Integrated DNA Technologies, Inc. as proposed for library preparation with TA ligation with ten different barcodes.

For sequencing, we used an Illumina MiSeq with a 151 bp single-end sequencing protocol.
The first Illumina files were written by the sequencer after 25 sequencing cycles, which was exactly 4 hours after starting the sequencing run.
30 minutes afterwards the innovative software analyzed all previous files and continued with live analysis. At that time, the reads were already assigned to the samples using the barcodes of the live sequencing adapters.
The first report was written 5:30 hours after starting the sequencing device (cycle 46) and included the results of 30 bp reads (not including random sequence and barcode).
The final analysis results were written 15 hours after starting the sequencing run for cycle 151 including the results for reads of length 133 right after the sequencing run finished.
Additional live reports were created after cycles 56, 66, 91 and 116 with negligible delay after the respective data was written by the sequencing device.

The results show that it was possible to detect the most relevant pathogens in all ten samples already with the first report written 5:30 hours after starting the sequencing run. With ongoing sequencing and real-time analysis, it was possible to get a complete picture of the microbes contained in the sample that went far beyond the identifications made by cultivation. The process of getting additional hits and higher confidence of the detected microbes also clearly shows the benefits of ongoing real-time analysis compared to a strict acceleration of the sequencing protocol for only the final results as more comprehensive results can be achieved with ongoing sequencing while the most important candidates are already present at early time points.

The following adapter oligonucleotides were used for the sequencing run (see Table 1), following the sequence design of the invention and differing only in the sequence of the barcode sequence between the different sequenced samples:

TABLE 1
Adapter oligonucleotide sequences used for parallel real-time analysis of ten samples.

| Sample | Barcode | SEQ ID NO | Adapter oligonucleotide sequences |
|---|---|---|---|
| A01 | GTCGTGAATC | SEQ ID NO: 1 | AATGATACGGCGACCACCGAGATCTACACTCTACACTCTTTCC |
| | (SEQ ID | | CTACACGACGCTCTTCCGATCTNNNNNGTCGTGAATC*T |
| | NO: 21) | SEQ ID NO: 2 | /5Phos/GATTCACGACNNNNNAGATCGGAAGAGCACACGTCT |
| | | | GAACTCCAGTCACTCTATCTCGTATGCCGTCTTCTGCTTG |
| A02 | CTGTTCTACG | SEQ ID NO: 3 | AATGATACGGCGACCACCGAGATCTACACTCTACACTCTTTCC |
| | (SEQ ID | | CTACACGACGCTCTTCCGATCTNNNNNCTGTTCTACG*T |
| | NO: 22) | SEQ ID NO: 4 | /5Phos/CGTAGAACAGNNNNNAGATCGGAAGAGCACACGTCT |
| | | | GAACTCCAGTCACTCTATCTCGTATGCCGTCTTCTGCTTG |
| A03 | GCTCTTAGGT | SEQ ID NO: 5 | AATGATACGGCGACCACCGAGATCTACACTCTACACTCTTTCC |
| | (SEQ ID | | CTACACGACGCTCTTCCGATCTNNNNNGCTCTTAGGT*T |
| | NO: 23) | SEQ ID NO: 6 | /5Phos/ACCTAAGAGCNNNNNAGATCGGAAGAGCACACGTCT |
| | | | GAACTCCAGTCACTCTATCTCGTATGCCGTCTTCTGCTTG |
| A04 | TAGAAGCCAT | SEQ ID NO: 7 | AATGATACGGCGACCACCGAGATCTACACTCTACACTCTTTCC |
| | (SEQ ID | | CTACACGACGCTCTTCCGATCTNNNNNTAGAAGCCAT*T |
| | NO: 24) | SEQ ID NO: 8 | /5Phos/ATGGCTTCTANNNNNAGATCGGAAGAGCACACGTCT |
| | | | GAACTCCAGTCACTCTATCTCGTATGCCGTCTTCTGCTTG |
| A05 | CACTTAGGTA | SEQ ID NO: 9 | AATGATACGGCGACCACCGAGATCTACACTCTACACTCTTTCC |
| | (SEQ ID | | CTACACGACGCTCTTCCGATCTNNNNNCACTTAGGTA*T |
| | NO: 25) | SEQ ID NO: 10 | /5Phos/TACCTAAGTGNNNNNAGATCGGAAGAGCACACGTCT |
| | | | GAACTCCAGTCACTCTATCTCGTATGCCGTCTTCTGCTTG |
| A06 | ATAGGTAAGG | SEQ ID NO: 11 | AATGATACGGCGACCACCGAGATCTACACTCTACACTCTTTCC |
| | (SEQ ID | | CTACACGACGCTCTTCCGATCTNNNNNATAGGTAAGG*T |
| | NO: 26) | SEQ ID NO: 12 | /5Phos/CCTTACCTATNNNNNAGATCGGAAGAGCACACGTCT |
| | | | GAACTCCAGTCACTCTATCTCGTATGCCGTCTTCTGCTTG |
| A07 | CGCCTTATAT | SEQ ID NO: 13 | AATGATACGGCGACCACCGAGATCTACACTCTACACTCTTTCC |
| | (SEQ ID | | CTACACGACGCTCTTCCGATCTNNNNNCGCCTTATAT*T |
| | NO: 27) | SEQ ID NO: 14 | /5Phos/ATATAAGGCGNNNNNAGATCGGAAGAGCACACGTCT |
| | | | GAACTCCAGTCACTCTATCTCGTATGCCGTCTTCTGCTTG |
| A08 | GCACGATGCT | SEQ ID NO: 15 | AATGATACGGCGACCACCGAGATCTACACTCTACACTCTTTCC |
| | (SEQ ID | | CTACACGACGCTCTTCCGATCTNNNNNGCACGATGCT*T |
| | NO: 28) | SEQ ID NO: 16 | /5Phos/AGCATCGTGCNNNNNAGATCGGAAGAGCACACGTCT |
| | | | GAACTCCAGTCACTCTATCTCGTATGCCGTCTTCTGCTTG |
| A09 | AATATGCCAG | SEQ ID NO: 17 | AATGATACGGCGACCACCGAGATCTACACTCTACACTCTTTCC |
| | (SEQ ID | | CTACACGACGCTCTTCCGATCTNNNNNAATATGCCAG*T |
| | NO: 29) | SEQ ID NO: 18 | /5Phos/CTGGCATATTNNNNNAGATCGGAAGAGCACACGTCT |
| | | | GAACTCCAGTCACTCTATCTCGTATGCCGTCTTCTGCTTG |
| A10 | TAGAGTCACG | SEQ ID NO: 19 | AATGATACGGCGACCACCGAGATCTACACTCTACACTCTTTCC |
| | (SEQ ID | | CTACACGACGCTCTTCCGATCTNNNNNTAGAGTCACG*T |
| | NO: 30) | SEQ ID NO: 20 | /5Phos/CGTGACTCTANNNNNAGATCGGAAGAGCACACGTCT |
| | | | GAACTCCAGTCACTCTATCTCGTATGCCGTCTTCTGCTTG |

As an example for the quality of pathogen identification results, the following results were produced with ongoing sequencing for sample A01 (see Table 2). The table shows the microbes found with the parallel live sequencing method of the invention. The different columns C46, C56, C66, C91, C116 and C151 show the results after cycle 46, 56, 66, 91, 116 and 151, respectively. The second row of the title indicates the elapsed time since the start of the sequencing device. “O” indicates a hit with low evidence. “X” indicates a hit with high evidence. The column “Cultivation” shows the identification results using a cultivation method for the same sample. “Clinically plausible” indicates the evaluation of a microbiological clinician whether the identified microbes were plausible for the given patient:

TABLE 2
Exemplary results of pathogen identification produced with ongoing sequencing for sample A01.

| | C46 | C56 | C66 | C91 | C116 | C151 | | Clinically |
|---|---|---|---|---|---|---|---|---|
| Microbe | (5:30 h) | (6:25 h) | (7:15 h) | (9:30 h) | (11:45 h) | (14:50 h) | Cultivation | plausible |
| Actinomyces sp. | ◯ | X | X | X | X | X | | X |
| Exophiala | ◯ | X | X | X | X | X | | X |
| dermatitidis | | | | | | | | |
| Haemophilus | X | X | X | X | X | X | | X |
| influenza | | | | | | | | |
| Neisseria sp. | X | X | X | X | X | X | | X |
| Prevotella sp. | ◯ | ◯ | X | X | X | X | | X |
| Rothia | X | X | X | X | X | X | | X |
| mucilagniosa | | | | | | | | |
| Staphylococcus | X | X | X | X | X | X | X | X |
| aureus | | | | | | | | |
| Streptococcus sp. | X | X | X | X | X | X | X | X |
| Veillonella sp. | ◯ | ◯ | X | X | X | X | | X |

The exemplary results for sample A01 show that the method of the invention can deliver reliable results already in early stages of sequencing, while evidence of the results increases with ongoing sequencing. Thereby, the method can identify a broader spectrum of microbes than what is usually found by alternative methods such as cultivation. The method of the invention additionally enables a significant enhancement of the diagnostic workflow when compared to standard Illumina sequencing processes by delivering reliable results even after a low fraction of the total number of sequencing cycles. Precisely, the first identification results with the method of the invention were achieved 09:20 hours before the sequencing run finished, i.e. before analysis of results can start in standard Illumina workflows.

### REFERENCES

[1] J. Quick, P. Ashton, S. Calus, C. Chatt, S. Gossain, J. Hawker, S. Nair, et al. “Rapid draft sequencing and real-time nanopore sequencing in a hospital outbreak of Salmonella.”, Genome Biology, pp. 16, 114, 2015.
[2] H. S. M. Engvall, K. Naess, N. Lesko, P. Larsson, M. Dahlberg, R. Andeer, et al. “Rapid pulsed whole genome sequencing for comprehensive acute diagnostics of inborn errors of metabolism.”, BMC Genomics., p. 15 (1): 1090, 2014.
[3] N. A. Miller, E. G. Farrow, M. Gibson, L. K. Willig, G. Twist, B. Yoo, T. Marrs, et al. “A 26-hour system of highly sensitive whole genome sequencing for emergency management of genetic diseases.”, Genome Medicine, p. 7:100, 2015.
[4] M. S. Lindner, B. Strauch, J. M. Schulze, S. H. Tausch, P. W. Dabrowski, A. Nitsche and B. Y. Renard. “HiLive: real-time mapping of illumina reads while sequencing”, Bioinformatics, pp. 917-919, 2017.
[5] T. P. Loka, S. H. Tausch, P. W. Dabrowski, A. Radonic, A. Nitsche and B. Y. Renard. “PriLive: privacy-preserving real-time filtering for next-generation sequencing”, Bioinformatics, pp. 34 (14): 2376-2383, 2018.
[6] S. H. Tausch, B. Strauch, A. Andrusch, T. P. Loka, M. S. Lindner, A. Nitsche and B. Y. Renard. “LiveKraken-real-time metagenomic classification of illumina data”, Bioinformatics, pp. 34 (21): 3750-3752, 2018.
[7] T. P. Loka, S. H. Tausch and B. Y. Renard. “Reliable variant calling during runtime of Illumina sequencing.”, Scientific Reports, p. 9:16502, 2019.
[8] S. H. Tausch, T. P. Loka, J. M. Schulze, A. Andrusch, J. Klenner, P. W. Dabrowski, M. S. Lindner, et al. “PathoLive-Real-time pathogen identification from metagenomic Illumina datasets”, BioRxiv, 2020.
[9] T. C. Glenn, R. A. Nilsen, T. J. Kieran, J. G. Sanders, N. J. Bayona-Vásquez, J. W. Finger, T. W. Pierson, et al. “Adapterama I: universal stubs and primers for 384 unique dual-indexed or 147,456 combinatorially-indexed Illumina libraries (iTru & iNext)”, PeerJ, 7: e7755, 2019.
D. W. Fadrosh, B. Ma, P. Gajer, N. Sengamalay, S. Ott, M. Brotman and R. J. Ravel. “An improved dual-indexing approach for multiplexed 16S rRNA gene sequencing on the Illumina MiSeq platform.”, Microbiome, p. 2 (1): 6, 2014. [11] J. Stahl, J. Myers, B. Culver, B. Kudlow. “Methods of nucleic acid sample preparation.” Patent WO 2018/053362 A1, 2018.
C.-H. Lin, G. Q. Zhao, S. Lin. “Cell-free nucleic acid standards and uses thereof.” Patent WO 2017/223366 A1, 2017.
J. Buis, R. D. Beaubien Jr., J. Stoerker. “Multimodal assay for detecting nucleic acid aberrations.” Patent WO 2018/094031 A1, 2018.

1. A method for real-time sequence analysis of DNA fragments, comprising:
providing at least one sample of DNA fragments for sequence analysis, connecting one kind of first and second adapter oligonucleotides to the 5′ and 3′ ends of a DNA strand of the DNA fragments of the sample, respectively, wherein:
a first adapter oligonucleotide comprises from 5′ to 3′:
a first flow cell binding sequence,
a read 1 sequencing primer site,
optionally a random sequence, and
a sample-specific barcoding sequence, and
a second adapter oligonucleotide comprises from 5′ to 3′:
a sequence complementary to the sample-specific barcoding sequence of the first adapter oligonucleotide,
optionally a sequence complementary to the random sequence,
a read 2 sequencing primer site, and
a second flow cell binding sequence,
wherein first and second adapter oligonucleotides of one kind have complementary barcoding sequences, and
sequencing of the DNA fragments comprising the connected adapter oligonucleotides in a sequencing by synthesis (SBS) process.

2. The method for real-time sequence analysis of DNA fragments according to claim 1, wherein:
the method is for parallel real-time analysis of DNA fragments from at least two samples,
at least two samples of DNA fragments are provided, and
for each sample a different kind of first and second adapter oligonucleotides are connected to the 5′ and 3′ ends of a DNA strand of the DNA fragments, wherein different kinds of adapter oligonucleotides have different barcoding sequences, and wherein the DNA fragments from the at least two samples comprising the connected first and second adapter oligonucleotides are sequenced in one reaction vessel, such as a flow cell.

3. The method according to claim 1, wherein the connecting of the adapter oligonucleotides occurs via ligation, amplification, tagmentation or combinations thereof.

4. The method according to claim 1, wherein the method comprises real-time data analysis during the sequencing process.

5. The method according to claim 4, wherein the data analysis during the sequencing process comprises:
the assignment of (preferably all) sequencing reads in the flow cell to the corresponding sample of DNA fragments based on the detected sample-specific barcoding-sequence;
provision of sample-specific data analysis results during the sequencing process, for example with respect to the presence of one or more specific DNA sequences in the sample;
evaluation of the reliability and completeness of real-time analysis results (i.e., results being reported before the end of the sequencing process) using algorithmic and statistical methods, learning-based approaches, artificial intelligence and/or combinations of these;
editing of the raw sequencing data, e.g. correcting detected sequencing errors and/or removing human reads from the raw sequencing data, for example to comply with data protection standards; and/or
the sample-specific visualization of analysis results during the sequencing process; wherein preferably the data analysis is performed by a computer program.

6. The method according to claim 1, wherein the method is used for:
the diagnosis of a medical condition, such as an infection and related antimicrobial resistances,
determining microbial compositions of a sample,
diagnosis or prognosis of an autoimmune disease, a transplant rejection reaction, a genetic disorder, or cancer;
the detection of genetically modified organisms; or
a forensic or hygiene analysis.

7. An adapter oligonucleotide for parallel real-time sequencing comprising from 5′ to 3′:
a first flow cell binding sequence, and
a read 1 sequencing primer site,
wherein 3′ (downstream) from the read 1 sequence primer site there is:
an optional random sequence, and
a sample-specific barcoding sequence.

8. The adapter oligonucleotide according to claim 7, wherein the adapter comprises 3′ (downstream) of the sequencing primer site and 5′ of the barcoding sequence a random sequence, wherein the random sequence has preferably a length of 3-10, more preferably 4-7 nucleotides.

9. The adapter oligonucleotide according to claim 7, comprising between the first flow cell binding sequence and the read 1 sequencing primer site an index or spacer sequence.

10. The adapter oligonucleotide according to claim 7, wherein the sample-specific barcoding sequence has a length of at least 4 nucleotides, preferably 4-16, more preferably 8-12 nucleotides.

11. The adapter oligonucleotide according to claim 7, wherein the adapter comprises at its 3′ end a connection site.

12. The adapter oligonucleotide according to claim 7, wherein:
the adapter oligonucleotide is hybridized to a second adapter oligonucleotide, and
the second adapter oligonucleotide comprises from 5′ to 3′:
optionally a connection site,
a sequence complementary to the sample-specific barcoding sequence, optionally a sequence complementary to the random sequence,
a read 2 sequencing primer site,
optionally an index or spacer sequence, and
a second flow cell binding sequence.

13. A kit for parallel real-time sequencing comprising:
a first adapter oligonucleotide for parallel real-time sequencing according to claim 7,
a second adapter oligonucleotide according to claim 7, wherein:
the second oligonucleotide is optionally hybridized to the adapter oligonucleotide, and
the second adapter oligonucleotide further includes from 5′ to 3′:
optionally a connection site,
a sequence complementary to the sample-specific barcoding sequence, optionally a sequence complementary to the random sequence,
a read 2 sequencing primer site,
optionally an index or spacer sequence, and
a second flow cell binding sequence,
optionally one or more reagents for connecting the adapter oligonucleotides to 5′ ends of DNA fragments comprised in a sample, and
and a computer program, preferably stored on a computer readable medium, for real time analysis of sequencing data generated in a sequencing process using the adapter oligonucleotides.

14. A use of the first adapter oligonucleotides and the second adapter oligonucleotides according to the kit of claim 13, in a method for real-time sequence analysis of DNA fragments.

15. The use according to claim 14, wherein the method enables real-time sequence analysis of DNA fragments and comprises:
providing at least one sample of DNA fragments for sequence analysis, connecting one kind of first and second adapter oligonucleotides to the 5′ and 3′ ends of a DNA strand of the DNA fragments of the sample, respectively, wherein the first and second adapter oligonucleotides of one kind have complementary barcoding sequences, and
sequencing of the DNA fragments comprising the connected adapter oligonucleotides in a sequencing by synthesis (SBS) process.
